(19) United States
(12) Patent Application Publication
(10) Pub. No.: US 2025/0121045 A1
(43) Pub. Date: Apr. 17, 2025

(54) DEVELOPMENT OF SYNTHETIC PSEUDAMINIC ACID-BASED ANTIBACTERIAL VACCINES AGAINST ACINETOBACTER BAUMANNII

(71) Applicants: Versitech Limited, Hong Kong (CN); City University of Hong Kong, Hong Kong (CN)

(72) Inventors: Xuechen LI, Hong Kong (CN); Ruohan WEI, Guangdong (CN); Han LIU, Hong Kong (CN); Sheng CHEN, Hong Kong (CN); Xuemei YANG, Hong Kong (CN)

(21) Appl. No.: 18/685,422

(22) PCT Filed: Jun. 21, 2022
(86) PCT No.: PCT/CN2022/100131

§ 371 Date: Feb. 21, 2024

Related U.S. Application Data
(60) Provisional application No. 63/260,512, filed on Aug. 23, 2021.

(51) Int. Cl. A61K 39/104; A61K 39/00; A61K 39/385; A61K 47/64; A61P 31/04

(52) U.S. Cl. CPC A61K 39/1045; A61K 39/385; A61K 47/646; A61P 31/04; A61K 2039/575; A61K 2039/6037; A61K 2039/6081

(57) ABSTRACT

Provided are compositions comprising chemically synthesized pseudaminic acid (Pse) conjugated to a carrier protein using the OPA chemistry, methods of using said compositions to stimulate immune responses in subjects and protect the vaccinated subjects from infections caused by Pse-producing A. baumannii.

### BACKGROUND OF THE INVENTION

Acinetobacter baumannii is a Gram-negative bacteria that can cause a range of infections in both the hospital and community, including bacteremia, pneumonia, meningitis, urinary tract infections, and skin and soft tissue infections¹. Predominantly, it is an opportunistic pathogen that can cause severe hospital-acquired infections especially among immunocompromised individuals². A. baumannii can also colonize in human without causing infections or symptoms, as well as exist widely in natural environments³. Bacterial resistance to multiple drugs is posing a global threat to the public health and severely affecting the effectiveness of public health management. A. baumannii has demonstrated the ability to acquire resistance to numerous classes of antibiotics via multiple resistance mechanisms⁴. A. baumannii is thought to exhibit extensive resistance to most last-line antibiotics in recent years, whereas it has been sensitive to most antibiotics before 1970s⁵. Carbapenems have served as last resort antibiotics to treat A. baumannii infections for years. However, the increasing trend of carbapenem resistance in A. baumannii has limited their efficacy and promoted the use of polymyxins and tigecycline as last-line drugs⁶. However, the emergence of A. baumannii resistant to colistin and tigecycline has now been reported, aggravating clinical problems caused by carbapenem-resistant (CR) A. baumannii^{7,8}. The treatment of A. baumannii infections has become difficult due to the emergence of multidrug-resistant strains, and the development of new strategies for preventing and treating infections caused by this pathogen is necessary. Among the 12 “priority pathogens” requiring urgent antibacterial research and development published by the World Health Organization (WHO) in 2017⁹, A. baumannii is on the top of this list as the top priority for immediate attention⁹.

Development of new antibacterial drugs against multi-drug resistant (MDR) A. baumannii has been continuingly pursued¹⁰. Apart from antibiotics, vaccination or immunotherapy is an alternative strategy to protect humans from bacterial infections and combat bacterial multidrug resistance¹¹. Over the past decades, a growing number of vaccine candidates against A. baumannii including whole bacteria, outer membrane vesicles or complexes, DNA-based vaccines and purified or recombinant subunits, have been proposed and studied¹². Bacterial surface carbohydrates have been established as effective antigens for vaccine development against infectious diseases^{13,14}. Glycoconjugate vaccines have been successfully developed and effectively used against Haemophilus influenzae type B¹⁵, selected serotypes of S. pneuomoniae¹⁶, Neisseria meningitidis serogroups A, C, W and Y¹⁷, and Salmonella typhi¹⁸. The structure of carbohydrate-based antibacterial vaccines is commonly composed of carbohydrate antigens, linkers and carrier proteins. The carbohydrate antigen can be the bacterial surface polysaccharide isolated from cultured bacteria, as in Prevnar 13® (Pfizer, New York, NY, approved by the FDA in 2010) containing cell capsule sugars of thirteen serotypes of S. pneumoniae (1, 3, 4, 5, 6A, 6B, 7F, 9V, 14, 18C, 19A, 19F and 23F) conjugated to diphtheria CRM197 carrier protein¹⁹. However, not all pathogens can be readily cultured and bacterial polysaccharide extraction can be plagued with contamination. Alternatively, synthetic carbohydrate antigens are structurally defined and free from cell-derived contaminants, as in Quimi-Hib® (CIGB, approved in 2004 in Cuba) containing synthetic polyribosylribitol phosphate conjugated to tetanus toxoid carrier protein²⁰. Various synthetic carbohydrate-based vaccine candidates against different pathogens are being explored²¹.

Pseudaminic acid (Pse), belonging to nonulosonic acid family, is widely distributed in numerous pathogenic bacteria as a component of repeating units constructing cell surface-associated glycans, such as lipopolysaccharide (LPS) in P. aeruginosa, Shigella boydii and Vibrio vulnificus, capsular polysaccharide (CPS) in A. baumannii, pili in P. aeruginosa, and flagella in Aeromonas caviae, H. pylori and Campylobacter jejuni²². Even though the exact function of Pse on bacterial cell surface remains unclear, it is likely to play an important role in bacterial pathogenicity as it is highly associated with virulence factors LPS, CPS, and flagella, and it is unique to Gram-negative bacteria and structurally similar to mammalian sialic acids. Together. CPS from pathogenic bacteria containing Pse is expected to be an effective target in generating vaccine against pathogens with Pse present on the surface. However, the development of Pse-based antibacterial vaccines has been hampered by not only the difficulty in extracting Pse-containing polysaccharide samples in high monodispersity and sufficient amount but also the lability of pseudaminyl linkage. Previously, Wu et al. reported that bacteriophage ΦAB6 tailspike protein is capable of specifically recognizing the exopolysaccharide (EPS) of A. baumannii strain 54149 and depolymerizing it to oligosaccharide fragment Pse5NAc7NAc-α-(2→6)-Glcp-β-(1→6)-[→3]-Galp-β-(1→3)-GalNAcp-β-(1]₂ as the major product²⁸. The resulting oligosaccharide was used in vaccinations after conjugated to the carrier protein, and the boosted sera from the vaccinated rabbit was shown to recognize EPS from A. baumannii strain 54149, but not EPS from A. baumannii strain SK44, which shares most sugar components with Ab-54149 other than Pse²⁸. These studies indicate the critical epitope of Pse on antigenicity. There remain considering challenges in creating A. baumannii such as antigen heterogeneity and batch-to-batch reproducibility encountered in isolating the CPS antigen from cultured bacteria and structural limitations based on the bacteriophage strategy, as naturally existing Pse forms diverse structures for pathogenic bacteria. Previous studies have also reported highly efficient synthesis of Pse and highly stereoselective pseudaminylation²⁹⁻³¹.

Therefore, there remains a need for safe and effective Acinetobacter baumannii vaccine.

### BRIEF SUMMARY OF THE INVENTION

The present invention relates to a novel, synthetic Pseudaminic acid (Pse)-based vaccine against Pse-bearing bacterial pathogens. In certain embodiments, Pse conjugated to a carrier protein are capable of stimulating immune responses. In certain embodiments, the Pse-carrier protein conjugate can protect vaccinated subjects from infections caused by Pse-bearing A. baumannii, particularly Pse-producing A. baumannii strain Ab2.

In certain embodiments, the ortho-phthalaldehyde (OPA) chemistry can be used to conjugate synthetic carbohydrates onto carrier proteins for glycoconjugate synthesis.

### BRIEF DESCRIPTION OF THE DRAWINGS

FIGS. 1A-1E Immunization schedule of Pse vaccines and antibody titers. (FIG. 1A) Ten C57BL/6J mice per group were immunized subcutaneously with three doses of Pse vaccines. Pse-CRM197 1, Pse-CRM197 2 and Pse-CRM197 3, mixed with aluminum hydroxide. Control mice received CRM197 mixed with aluminum hydroxide in PBS. Serum collected on day 7 (FIG. 1B), day 21 (FIG. 1C), day 35 (FIG. 1D) and day 65 (FIG. 1E) were two-fold diluted from 100 to determine the end point titer of Pse specific antibodies in post-immune sera analyzed by ELISA.

FIGS. 2A-2H isotyping of Pse specific antibodies in post-immune sera analyzed by ELISA. HRP conjugated goat anti-mouse IgA (FIG. 2A), IgM (FIG. 28). IgG1 (FIG. 2C), IgG2b (FIG. 2D). IgG2c (FIG. 2E), IgG3 (FIG. 2F), λ (FIG. 2G), and κ (FIG. 2H) were used to type the Pse specific antibodies.

FIG. 3 Flow cytometry analysis of binding capacity of post-immune sera toward A. baumannii strain Ab2. Bacteria were incubated with 100-diluted post-immune sera and Alexa Fluor 647-labeled secondary anti-mouse antibodies. The bacteria incubated with secondary antibodies only were used as a negative control.

FIGS. 4A-4C Immunization with the Pse vaccines protects against A. baumannii infection. (FIG. 4A) Determination of LD50 of A. baumannii strain Ab2 using a mice sepsis model. Survival of mice after intraperitoneal injection of the indicated dose of A. baumannii strain Ab2 (n=4-5 mice/group) was determined. Mice were vaccinated with the Pse vaccines at 0, 2 and 4 weeks and then challenged 2 weeks after the last immunization with 2.0×10⁷ CFU (2×LD50) (FIG. 41), and 5.0×10⁷ CFU (5×LD50) (FIG. 4C), of strain Ab2.

FIGS. 5A-5F Bacterial loads in liver (FIG. 5A), kidneys (FIG. 5B), lungs (FIG. 5C), spleens (FIG. 5D), hearts (FIG. 5E), and blood (FIG. 5F) from vaccinated and control mice at 12 h post infection (n=4 mice/group). Mice were vaccinated with the Pse vaccines at 0, 2 and 4 weeks and then challenged 2 weeks after the last immunization with 5.0×10⁷ CFU (5×LD50) of strain Ab2.

FIGS. 6A-6C Serum levels of proinflammatory cytokines of IL-1β (FIG. 6A), IL-6 (FIG. 6B), and TNF-α (FIG. 6C) from vaccinated and control mice at 12 h post infection (n=4 mice/group). Mice were vaccinated with the Pse vaccines at 0, 2 and 4 weeks and then challenged 2 weeks after the last immunization with 5.0×10 CFU (5×LD50) of strain Ab2, a, The level of IL-1β and TNF-α from mice vaccinated with Pse vaccines, Pse-CRM197 1, Pse-CRM197 2 and Pse-CRM197 3 were less than the lowest value (7.8 pg/ml) of the measurement range.

FIG. 7 Synthesis of Pse-CRM197 conjugates. Reagents and conditions: (a) NIS. TfOH, DMF, DCM, AW-300 molecular sieves, acceptor 6, −40° C., 6 h, 80%. (b) Pd/C, NH₄OAc, DCM-MeOH, H₂ (1 atm), 30 min, then NMM, Ac₂O, 1 h, 77%. (c) (i) DEA-MeCN, 30 min; (ii) acid 7, EDCI, DIPEA, DCM, 87%. (d) (i) LiOH, MeOH-THF-H₂O, 24 h; (ii) 10% HOAc (aq), 2 h, 75% over 2 steps. (e) CRM197 carrier protein. PBS buffer (pH 7.4), rt, 6 h. NIS: N-iodosuccinimide. TfOH: trifluoromethanesulfonic acid. DMF: N,N-dimethylformamide. DCM: dichloromethane. NMM: N-methylmorpholine. DEA: diethylamine. EDCI; 1-ethyl-3-(3-dimethylaminopropyl)carbodiimide. DIPEA: N,N-diisopropylethylamine. THF: tetrahydrofuran.

FIG. 8 Synthesis of BSA-Pse conjugates. Reagents and conditions: (a) NIS, TfOH, DMF, DCM, AW-300 molecular sieves, acceptor 12, −40° C., 6 h, 82%. (b) Pd % C, NH₄OAc, DCM-MeOH, H₂ (latm), 30 min. then NMM. Ac₂O, 1 h, 78%. (c) (i) DEA-MeCN, 30 min; (ii) acid 13, EDCI, DIPEA, DCM, 87%. (d) LiOH, MeOH-THF-H₂O, 24 h, 79%. (e) sulfo-EMCS, PBS (pH 8.0), r.t., 2 h. (f) PBS (pH 7.4), r.t., 16 h. NIS: N-iodosuccinimide. TfOH: trifluoromethanesulfonic acid. DMF: N,N-dimethylformamide. DCM: dichloromethane. NMM: N-methylmorpholine. DEA: diethylamine. EDCI: 1-ethyl-3-(3-dimethylaminopropyl)carbodiimide. DIPEA: N,N-diisopropylethylamine. THF: tetrahydrofuran. Sulfo-EMCS: N-(ε-maleimido caproyloxy)sulfosuccinimide ester.

FIG. 9 Synthesis of (9H-fluoren-9-yl)methyl N-(2-(242-(2-(4,8-di-O-acetyl-5-azido-7-N-benzyloxycarbonyl-1-isopropyl-α-pseudaminosyloxy)ethoxy)ethoxy)ethoxy)ethyl)carbamate (6).

FIG. 10 Synthesis of (9H-fluoren-9-yl)methyl N-(2-(2-(2-(2-(5,7-di-acetamido-4,8-di-O-acetyl-1-isopropyl-α-pseudaminosyloxy)ethoxy)ethoxy)ethoxy)ethyl)carbamate (7).

FIG. 11 Synthesis of 3-(1,3-dimethoxy-1,3-dihydroisobenzofuran-5-yl)-N-(2-(2-(2-(2-(5,7-di-acetamido-4,8-di-O-acetyl-1-isopropyl-β-pseudaminosyloxy)ethoxy)ethoxy)ethoxy)ethyl)propanamide (9).

FIG. 12 Synthesis of 3-(3,4-diformylphenyl)-N-(2-(2-(2-(5,7-di-acetamido-α-pseudaminosyloxy)ethoxy)ethoxy)ethoxy)ethyl)propanamide (10).

FIG. 13 Synthesis of (9H-fluoren-9-yl)methyl N-(4,8-di-O-acetyl-5-azido-7-N-benzyloxycarbonyl-1-isopropyl-α-pseudaminosyloxy)pentanylcarbamate (12).

FIG. 14 Synthesis of (9H-fluoren-9-yl)methyl N-(5,7-di-acetamido-4,8-di-O-acetyl-1-isopropyl-α-pseudaminosyloxy)pentanylcarbamate (13).

FIG. 15 Synthesis of S-(2-((5-(5,7-di-acetamido-4,8-di-O-acetyl-1-isopropyl-α-pseudaminosyloxy) pentanyl)amino)-2-oxoethyl) ethanethioate (15).

FIG. 16 Synthesis of 2-mercapto-N-(5-(5,7-di-acetamido-α-pseudaminosyloxy)pentyl)acetamide (16),x

FIGS. 17A-17J SDS-PAGE of CRM197 before reaction (FIG. 17A), conjugate with 20, 30, 50 equivalents of Pse respectively. SDS-PAGE and Western Blot (using anti α-Pse antibody) of BSA (FIGS. 17B-17C) before reaction, after activation and after conjugation. (FIGS. 17D-17J), MALDI-TOF analysis of Pse-protein conjugates to measure the average molecular size. The recombinant CRM197 and BSA were measured as the standard.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising” “comprises”, “comprise” “consisting essentially of” “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X: 10%). As is apparent, this variation represents a range that is up to 10% above or below a given value, for example, X±1%, X±2%, X±3%. X±4%, X±5%, X±6%, X±7%,X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a vaccine. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder: immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject or that is capable of providing enhancement in health or function to the immune system or an organ, tissue, or body system. In other words, when administered to a subject, the amount is “therapeutically effective.” The actual amount will vary depending on a number of factors including, but not limited to, the particular condition, disease, or disorder being treated, prevented, or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to enhance immune system function and/or prevent or treat a bacterial infection. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

Pseudaminic acid (Pse) Conjugate Compositions and Methods of Using the Compositions

The subject invention pertains to methods of raising an immune response against Pse-producing bacteria, such as, for example. Acinetobacter baumannii. In some embodiments, the method comprises administering a composition to the subject, wherein the composition comprises a Pse conjugated to an immunogenic carrier protein.

The composition may be administered to a subject that does not have a bacterial infection at the time of administration, as prophylaxis, to prevent or delay the onset of a bacterial infection. The composition may also be administered to a subject that does have a bacterial infection at the time of administration, as therapy, to alleviate or eliminate one or more symptoms of the infection.

In certain embodiments, a glycoconjugate construct may be administered to a subject to raise an immune response in the subject, the method comprising Pseudaminic acid (Pse) conjugated to a carrier protein.

In certain embodiments, a composition that may be administered to a subject to raise an immune response in the subject, comprising Pse conjugated to a carrier protein. In certain embodiments, a pharmaceutical carrier, excipient, and/or adjuvant can be used in the composition.

In some embodiments, the target Acinetobacter spp., specifically Acinetobacter baumannii and strains thereof, including, for example A. baumannii strain Ab2 or other A. baumannii strains containing Pse, such as, for example, the K2, K6. K16. K23, K31, K33, K42. K46, K58, K77, K81, K90, K93 and K120 serotypes.

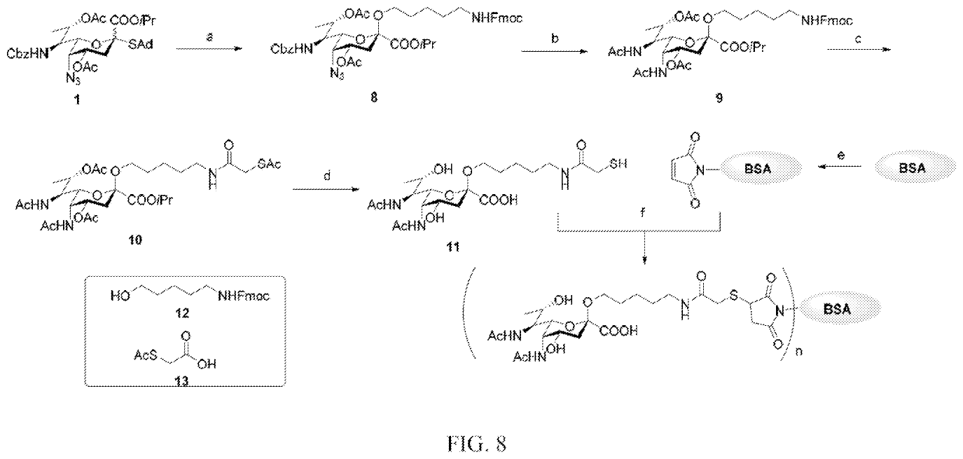

In certain embodiments, an ortho-phthalaldehyde (OPA)-Pseudaminic acid linker can be synthesized. In certain embodiments, the OPA can be used to react with primary amines to conjugate Pseudaminic acid to the carrier protein. In certain embodiments, the Pse donor, such as, for example, Pse donor 4, can be stereoselectively glycosylated with an Fmoc-protected PEG linker 5 in 80% yield (FIG. 7). In certain embodiments, the resulting N5-azide and N7-benzyl carbamate 6 can be converted to acetamide 7 by hydrogenolysis and acetylation. The Fmoc group can then be removed to generate a free amine (using diethylamine or other secondary amine reagents such as, for example, piperidine and 4-methylpiperidine) that can be coupled with an acid (FIG. 8), such as, for example, acid 8 containing methyl acetal protected form of phthaldehyde. Finally, deprotection was conducted by treating 9 with lithium hydroxide (or, for example, potassium hydroxide, sodium hydroxide, tetra-n-butylammonium hydroxide, and other hydroxide sources) followed by 10% or other concentrations ranging from about 5% to about 75% aqueous acetic acid solution to give rise to the Pse-OPA moiety, such as, for example Pse-OPA moiety 10. Thus, the Pse-OPA moiety subsequently reacted with a carrier protein, such as, for example, CRM197, in phosphate buffered saline (PBS, pH 7.4) to generate CRM197-Pse conjugate. In certain embodiments, the carrier protein can be diphtheria toxoid (DT), tetanus toxoid (TT), meningococcal outer membrane protein complex (OMPC). H. influenzae protein D (HiD), keyhole limpet hemocyanin (KLH), bovine serum albumin (BSA), or human serum albumin (HSA).

In certain embodiments, the Pse conjugate can be synthesized using about 1 to about 50, about 2 to about 50, about 3 to about 50, about 4 to about 50, about 5 to about 50, about 10 to about 50, about 20 to about 50, about 30 to about 50, about 20, about 30, or about 50 equivalent OPA-Pse moiety to generate a Pse-carrier protein compounds. In certain embodiments, the Pse-carrier protein compound can be Pse-CRM197 1 (sugar/protein ratio: 4.76), Pse-CRM197 2 (sugar/protein ratio: 8.27), or Pse-CRM197 3 (sugar/protein ratio: 14.34), as measured by MOLDI-TOF mass spectrometry.

In certain embodiments, the compound of the subject invention is provided by formula (I).

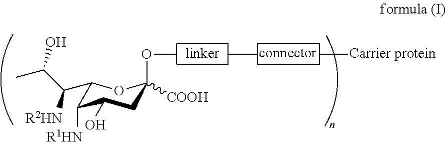

The compounds of the subject invention can comprise at least one pseudaminic acid (Pse) moiety conjugated to a carrier protein, wherein the pseudaminic acid moiety can be linked to the carrier protein by a linker and/or a connector. In certain embodiments, the R¹ group on the N5 position can be acetyl, formyl, or (R)-3-hydroxybutyryl. In certain embodiments, the R² group on the N7 position can be acetyl, formyl, or (R)-3-hydroxybutyryl. In certain embodiments, the linkage between the pseudaminic acid moiety and the linker is a glycosidic linkage, including is a or (S linkage.

In certain embodiments, the compound of the subject invention can have a PEG-based linker with variable number of (CH₂CH₂O) units, according to formula (H), wherein m is about 1 to about 5.

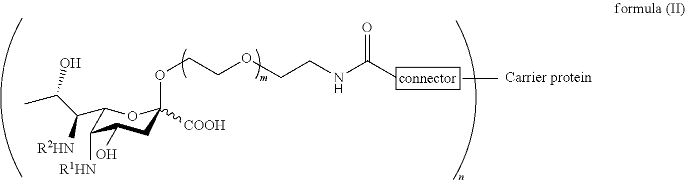

In certain embodiments, the compound of the subject invention can have a linker that can be a saturated hydrocarbon chain with variable length of about 2 to about 10, according to formula (III). The m value can range from about 0 to about 8.

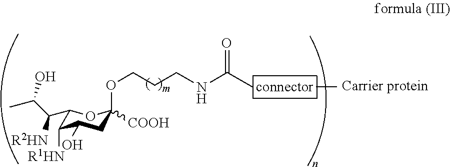

In certain embodiments, the compound of the subject invention can have a connector that can be cyclic lactam structure, optionally generated via the ligation between orthophthaldehyde (OPA) moiety and the lysine side chain, according to formula (IV). The m value can range from about 0 to about 5.

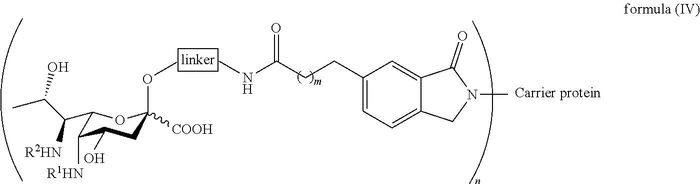

In certain embodiments, the compound of the subject invention can have a connector that can be a maleimide thiol adduct, optionally generated via Michael addition of thiol to the maleimide modified lysine side chain, according to formula (V). In certain embodiments, the lysine side chain maleimide modification can be installed via SMCC or other reagents containing maleimide and amine-reactive NHS ester. The m value can range from about 1 to about 5.

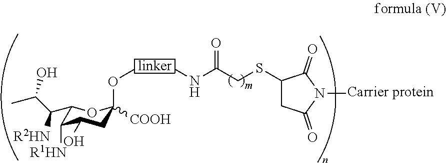

In certain embodiments, the compound of the subject invention can have a connector that can be a triazole-based structure, optionally generated from the sugar derived alkyne species and azide modified protein side chain, according to formula (VI). The azide modification can be installed onto the lysine side chain of the carrier protein using azide containing NHS ester or other active esters. The m value can range from about 0 to about 5, while the p value can range from about 1 to about 5, formula (VI):

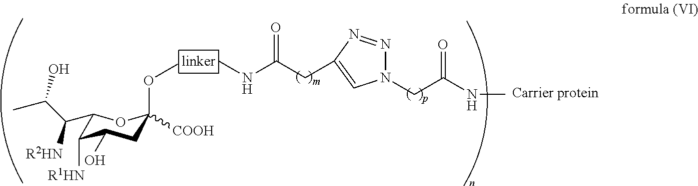

In certain embodiments, the compound of the subject invention can have a connector that can be a triazole-based structure, optionally generated from the sugar derived azide species and alkyne modified protein side chain, according to formula (VII). The alkyne modification can be installed onto the lysine side chain of the carrier protein using alkyne containing NHS ester or other active esters. The m value can range from about 1 to about 5, while the p value can range from about 0 to about 5.

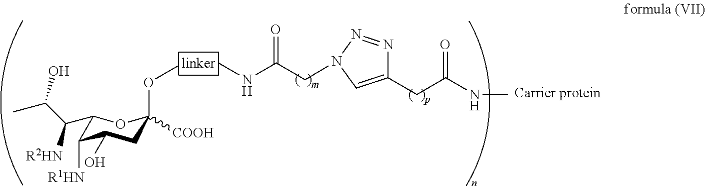

In certain embodiments, the compound of the subject invention can have a connector that can be a thiol-alkyne adduct, optionally generated via radical addition, according to formula (VIII). The alkyne modification can be installed onto the lysine side chain of the carrier protein using alkyne containing NHS ester or other active esters. The m value can range from about 1 to about 5, while the p value can range from about 0 to about 5.

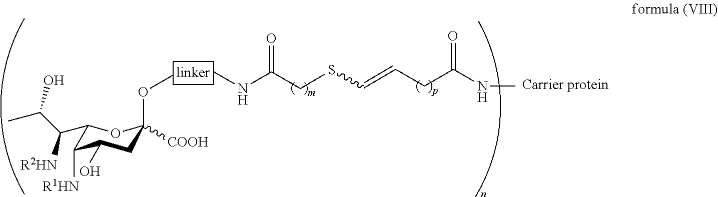

In certain embodiments the compound of the subject invention is Pse-C %%197 1 (formula (IX)), Pse-CRM197 2 (formula (X)), Pse-CRM197 3 (formula (XI)), Pse-BSA 17 (formula (XII)), or other compounds with variations at the sites described above:

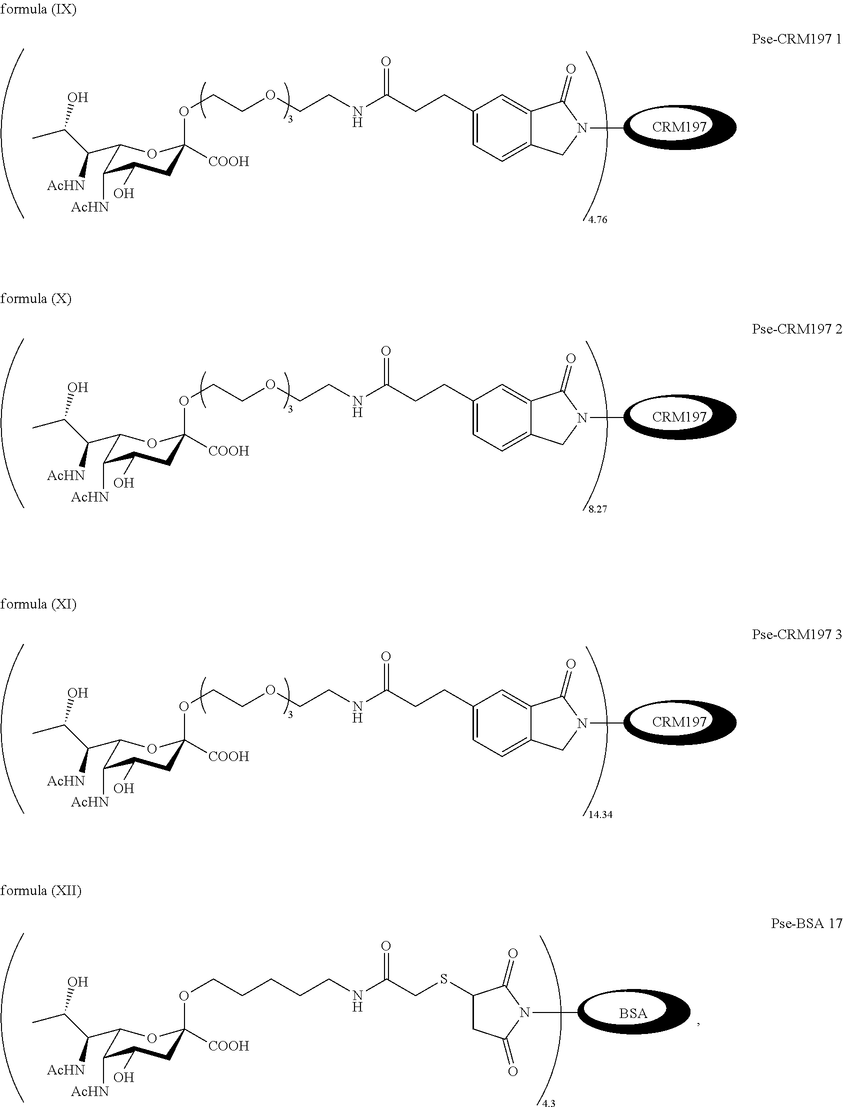

In some embodiments, the composition further comprises a suitable carrier, diluent, or buffer. Compositions contemplated within the scope of the invention can comprise one or more other compounds for raising an immune response and/or for therapy or prophylaxis for a bacterial infection. For example, a Pse-carrier protein conjugate of the invention can be provided in a composition with one or more of adjuvants and/or antibiotics. In one embodiment, the composition comprises the Pre-carrier protein conjugate in a pharmaceutically or physiologically acceptable carrier, buffer, or diluent.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea. Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65. Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols. DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

The Pse-carrier protein conjugates of the present invention can be formulated into pharmaceutically-acceptable salt forms. Pharmaceutically-acceptable salts of the Pse-carrier protein conjugates of the invention can be prepared using conventional techniques. “Pharmaceutically acceptable salt” includes both acid and base addition salts. A pharmaceutically acceptable salt of any one of the Pse-carrier protein conjugates described herein is intended to encompass any and all pharmaceutically suitable salt forms. Preferred pharmaceutically acceptable salts described herein are pharmaceutically acceptable acid addition salts and pharmaceutically acceptable base addition salts. In some embodiments, the pharmaceutically acceptable salt comprises acetate, chloride, or trifluoroacetic acid (TFA) salt.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Therapeutic and prophylactic application of the subject Pse-carrier protein conjugates and compositions thereof can be accomplished by any suitable therapeutic method and technique presently or prospectively known to those skilled in the art. The Pse-carrier protein conjugates can be administered by any suitable route known in the art including, for example, topical, oral, mucosal (e.g., nasal), rectal, parenteral, subcutaneous, or intravascular (e.g., intravenous) routes of administration. Thus, administration can be local at a desired anatomical site on the subject (e.g., a site of current infection or potential infection) or systemic. Administration of the Pse-carrier protein conjugates of the invention can be continuous or at distinct intervals as can be readily determined by a person skilled in the art.

The Pse-carrier protein conjugates and compositions of the subject invention can be administered to a subject with one on or more adjuvants. The adjuvant may be administered simultaneously or consecutively with the Pse-carrier protein conjugates and compositions thereof. Adjuvants may be administered within the same composition as the Pse-carrier protein conjugates or in a separate composition. In some embodiments, the adjuvant is an alum salt or other mineral adjuvant, bacterial product or bacteria-derived adjuvant, tensoactive agent (e.g., saponin), o/w or w/o emulsion, liposome adjuvant, cytokine (e.g., IL-2, GM-CSF, IL-12, and IFN-gamma), alpha-galactosylceramide analog, or toll-like receptor (TLR) ligand. In another embodiment, the adjuvant is QS21. Freund's complete or incomplete adjuvant, aluminum phosphate, aluminum hydroxide. BCG or alum. Further specific examples of adjuvants are provided in Pasquale A D et al., “Vaccine Adjuvants: from 1920 to 2015 and Beyond”, Vaccines, 2015, 3:320-343: Petrovsky N. et al., “Vaccine Adjuvants: Current State and Future Trends,” Immunology and Cell Biology, 2004, 82:488-496; and Vogel F R. “Improving Vaccine Performance with Adjuvants,” Clin Infect Dis., 2000, 30 (Supplement 3): S266-S270, which are incorporated herein by reference in its entirety.

The Pse-carrier protein conjugates and compositions of the subject invention can also be administered utilizing liposome technology, slow release capsules, implantable pumps, and biodegradable containers. These delivery methods can, advantageously, provide a uniform dosage over an extended period of time.

Pse-carrier protein conjugates can be covalently bound or otherwise linked to molecules that increase the half-life, solubility, bioavailability, or immunogenicity of an antigen (e.g., an adjuvant). Molecules that may be covalently bound to the antigen include a carbohydrate, biotin, poly(ethylene glycol) (PEG), polysialic acid, N-propionylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the naturally produced form of a protein is covalently bound to a moiety that stimulates the immune system.

The subject invention also concerns a packaged dosage formulation comprising in one or more containers at least one Pse-carrier protein conjugates and/or composition of the subject invention formulated in a pharmaceutically acceptable dosage. The package can contain discrete quantities of the dosage formulation, such as tablet, capsules, lozenge, and powders. The quantity of Pse-carrier protein conjugates in a dosage formulation and that can be administered to a patient can vary from about 1 mg to about 5000 mg, or about 1 mg to about 2000 mg, or more typically about 1 mg to about 500 mg, or about 5 mg to about 250 mg, or about 10 mg to about 100 mg.

The subject invention also concerns kits comprising one or Pse-carrier protein conjugates, compositions, compounds, or molecules of the present invention in one or more containers. In one embodiment, a kit contains a Pse-carrier protein conjugates and/or composition of the present invention.

A kit of the invention can also comprise, in addition to a Pse-carrier protein conjugates and/or composition of the invention, one or more compounds, biological molecules, or drugs for treating a pathogenic infection such as an A. baumannii infection.

In one embodiment, a kit of the invention includes instructions or packaging materials that describe how to administer Pse-carrier protein conjugates, compositions, compounds, or molecules of the kit. Containers of the kit can be of any suitable material, e.g., glass, plastic, metal, etc., and of any suitable size, shape, or configuration. In one embodiment, Pse-carrier protein conjugates, compositions, compounds, or molecules of the invention is provided in the kit as a solid, such as a tablet, pill, or powder form. In another embodiment, Pse-carrier protein conjugates, compositions, compounds, or molecules of the invention are provided in the kit as a liquid or solution, in one embodiment, the kit comprises an ampoule or syringe containing a Pse-carrier protein conjugates, compositions, compounds, or molecules of the invention in liquid or solution form. In one embodiment, the kit further includes one or more adjuvants, such as those disclosed herein.

Any methods of the subject invention can optionally include a step of identifying a person or animal who is or who may be in need of treatment or prevention of a disease, disorder, or condition (e.g., A. baumannii infection).

Biological samples refer to a fluid or tissue composition obtained from a human or animal. Biological samples within the scope of the invention include, but are not limited to, cells, whole blood, peripheral blood, blood plasma, bone marrow, spleen, serum, urine, tears, saliva, sputum, exhaled breath, nasal secretions, pharyngeal exudates, bronchoalveolar lavage, tracheal aspirations, interstitial fluid, lymph fluid, meningeal fluid, amniotic fluid, glandular fluid, feces, perspiration, mucous, vaginal or urethral secretion, cerebrospinal fluid, and transdermal exudate. A biological sample also includes experimentally separated fractions of all of the preceding solutions or mixtures containing homogenized solid material, such as feces, cells, tissues, and biopsy samples.

In certain embodiments, the Pse-carrier protein conjugates can elicit humoral responses. In certain embodiments, Pse-carrier protein conjugates can elicit the production of IgG1, IgG2b, IgG3, and IgG2c.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Materials and Methods

Bacterial Strains and Mice Used in this Study

Pseudaminic Acid producing A. baumannii strain Ab2 were reported previously²⁹. Bacteria were cultured in Luria-Bertani (LB) broth or Brain heart infusion (BHI) agar at 37° C.

Male C57BL/6J inbred strains of mice (six to eight weeks old, ˜20 g) were obtained from the Laboratory Animal Research Unit (LARU), City University of Hong Kong. Animals were rested and handled in strict accordance with the Animals (Control of Experiments) Ordinance (Cap. 340), Hong Kong. All animal experiments were approved by the Animal Research Ethics Sub-Committee (ARESC) of City University of Hong Kong. Animals were housed under specific pathogen-free conditions during experiments. All efforts were made to minimize the animal suffering.

### Immunization of Mice

Male 6-8 weeks old inbred C57BL/6J mice were immunized subcutaneously (s.c.) with Pse vaccines (2.4 μg sugar per dose) mixed with 1:1 (v/v) aluminum hydroxide (Thermo Fisher Scientific. Waltham. MA, United States). The control mice received CRM197 mixed with aluminum hydroxide in PBS. On day 14 and 28 mice received a booster injection with the same formulation. Blood (50 μl) was withdrawn on days 0, 21, 35 and 65 from the tail vein and centrifuged (5000× g, 10 min, room temperature) to retrieve serum. The antibody responses were measured in sera using ELISA.

### ELISA Assays to Detect the Immunogenicity of the Vaccine

High binding, 96 well polystyrene microtiter plates were coated overnight at 4° C. with BSA-Pse (2 μg/ml, 100 μl per well) in a sodium carbonate-bicarbonate buffer. pH 9.6. The next day, the plates were washed thrice with PBS containing 0.1% Tween-20 (PBST) and blocked with 2% BSA-PBS (200 μl per well) at 37° C. for 2 h. After washing thrice with PBST, the plates were incubated with post-immune sera (100 μl per well) in two-fold dilutions starting from 100 at 37° C. for one hour. Again, the plates were washed thrice with PBST and further incubated with horseradish peroxidase (HRP) conjugated goat anti-mouse antibody (Abcam. Cambridge. United Kingdom), used in 1:10000 dilutions in PBS (100 μl per well) followed by an incubation at 37° C. for 1 h. The plates were washed thrice with PBST and developed using tetramethylbenzidine (TMB). The reaction was stopped by adding 2% sulfuric acid and the absorbance was recorded at 450 nm. The Pse specific mouse antibody isotyping was performed using the SBA Clonotyping System for C57BL/6 mouse (SouthernBiotech, Birmingham, AL, United States). BSA-Pse was used to coat the 96 well polystyrene microtiter plates and capture the Pse specific antibodies in sera. HRP conjugated goat anti-mouse IgA. IgG1, IgG2b, IgG2c, IgG3, IgM, κ, and λ were used to type the Pse specific antibodies.

### Flow Cytometry

Flow cytometry analysis was performed to determine the binding capacity of post-immune sera toward Pse producing A. baumannii strain. Briefly, overnight culture of A. baumannii strain Ab2 was collected, washed with PBS and adjusted to OD˜0.2 using PBS. 500 μl of a bacterial suspension was incubated with 100-diluted post-immune sera collected on day 35 for 1 h. After washing with PBS, the bacteria were incubated with Alexa Fluor 647-labeled secondary goat anti-mouse antibody (Abcam) for 1 h. After further washing, the bacteria were resuspended in 2 ml PBS and then analyzed by BD FACSVia flow cytometry (BD Biosciences. Franklin Lakes, NJ). The bacteria incubated with secondary antibodies only were used as negative control.

### Efficacy of Pse Based Vaccine

A mouse sepsis model was used to characterize the efficacy of the Pse vaccines treatment³². Firstly, fifty percent lethal dose (LD₅₀) values for A. baumannii strain Ab2 were determined by infecting mice with serially diluted bacteria. A. baumannii strain Ab2 was cultured to logarithmic phase (OD˜0.6) at 37° C. in LB medium and then adjusted to the appropriate concentration in PBS. Bacterial concentrations of the inoculum were determined by plating on BHI agar plates. Male 6-8 weeks old inbred C57BL/63 mice were infected intraperitoneally with 0.2 ml of the bacterial suspension. Survival rate of mice were observed and recorded for 7 days post infection at 12 hours intervals. Survival rates of vaccinated mice were determined by infecting mice with A. baumannii strain Ab2. Mice were immunized as previously described, at day 0, 14 and 28. Two weeks after the final immunization, vaccinated and control mice in groups of 4 were inoculated with A. baumannii strain Ab2 at a high concentration, namely two times of LD50 (2×LD50) and five times of LD₅₀ (5×LD₅₀), respectively. Survival rate of mice were observed and recorded for 7 days post infection. Appearances and behaviors were also evaluated.

Post-infection tissue bacterial loads were determined for vaccinated and control mice at a high bacterial load of 5×LD50 of A. baumannii strain Ab2. Mice were anesthetized at 12 h after inoculated of bacteria. Blood and tissues including spleen, kidney, lung, liver and heart were removed aseptically. Tissues were weighed and then homogenized in sterilized PBS. Serial dilutions of tissues and blood were plated on BHI agar and incubated at 37° C. for bacterial quantification. Serum levels of interleukin-1β (IL-1β), tumor necrosis factor alpha (TNF-α), and IL-6 were determined in mice at 12 h post infection of 5×LD50 of A. baumannii strain Ab2, using mouse ELISA kits (Thermo Fisher Scientific).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1-Synthesis of Pse-Based Antibacterial Vaccines

As carbohydrates are T-cell independent antigens that cannot generate strong, long-lasting and memorable IgG antibody, conjugation with immunogenic carrier protein capable of activating helper T cells is necessary to enhance the anti-glycan antibody titer. The non-toxic mutant of diphtheria toxin, CRM197, is currently widely used as the carrier protein for glycan to make them immunogenic. We designed an ortho-phthalaldehyde (OPA)-pseudaminic acid liner, where OPA was reported to react with primary amines, to conjugate pseudaminic acid to the carrier protein. To this end, the Pse donor 4 was stereoselectively glycosylated with Fmoc-protected PEG linker 5, giving the α-glycoside 6 in 80% yield (FIG. 7). The glycosylation condition used here was NIS/TfOH in DCM using DMF as additive. Other activation conditions, such as NIS/TMSOTf, NIS/AgOTf, TolSCl/AgOTf and PhSCl/AgOTf can be used alternatively, giving less variable yields and selectivity. The additive DMF can be changed to other amide species such as N-formylpiperidine and N,N-dimethylacetamide. After converting N5-azide and N7-benzyl carbamate to acetamide 7 by hydrogenolysis and acetylation. Fmoc group was then removed to generate a free amine which was coupled with acid 8 containing methyl acetal phthaldehyde (FIG. 8). The coupling condition used here (EDCI/DIPEA) can be changed to other amide coupling conditions such as DCC, DIC, HATU, HBTU, PyBOP, PyBroP, DEPBT. EEDQ, and COMU. Finally, deprotection was conducted by treating 9 with lithium hydroxide followed by 10% aqueous acetic acid solution (or other concentrations ranging from 5% to 75%) to give rise to the Pse-OPA moiety 10. Thus, the Pse-OPA moiety subsequently reacted with CRM197 carrier protein in phosphate buffered saline (PBS, pH 7.4) to generate CRM197-Pse conjugate. In order to explore the difference in immune response caused by different antigen loading, we synthesized the conjugate using 20, 30, 50 equivalent OPA-Pse moiety to generate Pse-CRM197 1 (sugar/protein ratio: 4.76), Pse-CRM197 2 (sugar/protein ratio: 8.27), and Pse-CRM197 3 (sugar/protein ratio: 14.34), respectively.

In addition, we also synthesized Bovine serum albumin (BSA)-Pse conjugate 17 as the surrogate of natural glycan to verify the anti-Pse antibody generated by the vaccine. A different alkyl linker and thiol-maleimide strategy was used in order to diminish the unexpected recognition of the carrier protein and linker to the boosted sera. Compound 13 was obtained using the same strategy as described above using Pse donor 4 and FmocNH(CH₂)₆OH 11, and 2-(acetylthio)acetic acid 14 was coupled after Fmoc removal to give 15 (FIG. 8). Finally, the Pse-thiol linker 16 was obtained after saponification. Commercially available BSA was treated with N-(εmaleimidocaproxy)sulfosuccimide ester (sulfo-EMCS) in PBS (pH 7.4) to install maleimide on the protein, which further reacted with Pse-thiol linker 16 in PBS (pH 7.4) to give BSA-Pse conjugate 17.

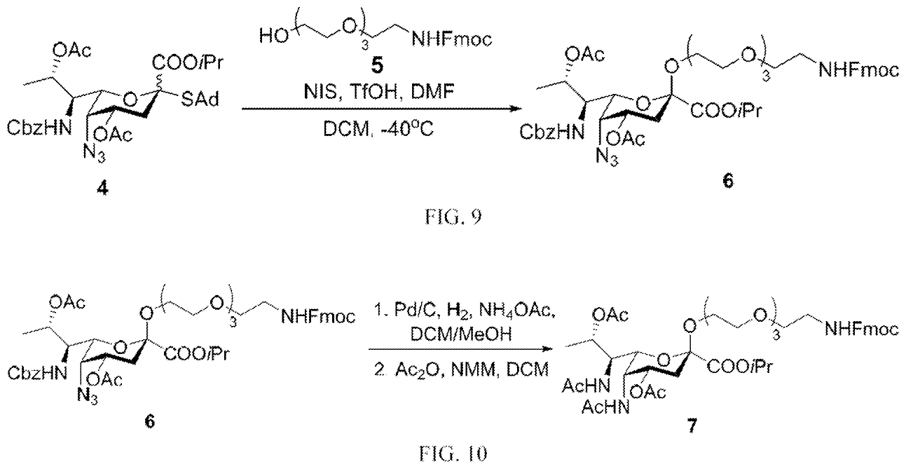

### Synthesis of (9H-fluoren-9-yl)methyl N-(2-(2-(242-(4,8-di-O-acetyl-5-azido-7-N-benzyloxycarbonyl-1-isopropyl-α-pseudaminosyloxy)ethoxy)ethoxy)ethoxy)ethyl)carbamate (6) (FIG. 9)

To a flame-dried Schlenk tube, flame-dried AW-300 molecular sieves (100 mg) were added under argon, followed by Pse donor 4 (41.7 mg, 0.0500 mmol, 1.0 equiv), acceptor 5 (46.0 mg, 0.100 mmol, 2.0 equiv), anhydrous DCM (freshly distilled over CaH₂, 1.0 mL), and anhydrous DMF (17.0 μL, 0.250 mmol, 5.0 equiv). After being stirred at r.t. for 1 h, the mixture was cooled to −78° C., and N-iodosuccinimide (27.0 mg, 0.120 mmol, 2.4 equiv) was added. Finally, triflic acid (0.450 μL, 0.1 equiv) was added dropwise to initiate the reaction, and the mixture was stirred at −40° C. for 8 h. Upon full conversion, as indicated by TLC, the reaction was quenched by addition of Et₆N. The mixture was diluted with ethyl acetate and filtered through celite. The organic phase was subsequently washed with sat. NaHCO₃ (aq), dried over anhydrous Na₂SO₄ and concentrated under vacuum. The product 6 was purified by silica gel column chromatography using n-hexane:ethyl acetate 1:1 v/v as eluent. R_{f}(n-hexane:ethyl acetate 1:1)=0.15. Only the a anomer was obtained (J_{Cl-Hz}=0 Hz, measured on Advance DRX Bruker 500 MHz NMR spectrometer by non-decoupled ¹³C spectroscopy) as a white solid (37.3 mg, 80%).

¹H NMR (500 MHz, CDCl₃): δ=7.75 (d, J=7.5 Hz, 2H, ArH), 7.52-7.68 (m, 2H, ArH), 7.39 (t, J=7.5 Hz, 2H, ArH), 7.26-7.36 (m, 7H, ArH), 5.59 (d, J=10.0 Hz, 1H, NH), 5.30-5.41 (m, 2H, H-4, NH), 5.00-5.15 (m, 3H. PhCH₂O, CH(CH₃)₂), 4.33-4.45 (m, 3H, H-7, C₁₂H₈CHCH₂), 4.20 (t, J=6.5 Hz, 1H, C₁₂H₈CHCH₂), 4.11 (d, J=9.5 Hz, 1H, H-6), 3.90 (s, 11H, H-5), 3.76-3.84 (m, 1H, OCH₂CH₂O), 3.53-3.70 (m, 10H, OCH₂CH₂O), 3.36-3.52 (m, 4H, OCHC₁₂O), 3.26-3.31 (m, 1H, NHCH₂), 2.13-2.17 (m, 2H, H-3a, H-3e), 2.10 (s, 3H, CH₃CO), 2.02 (s, 3H, CH₃CO), 1.35 (d, J=6.5 Hz, 3H, H-9), 1.27 (d, J=6.0 Hz, 3H. CH(CH₃)₂), 1.27 (d, J=6.0 Hz, 3H, CH(CH₃)₂).

¹³C NMR (125 MHz, CDCl₃): δ=170.8, 170.3, 166.4, 16.4, 156.7, 156.2, 144.2, 141.4, 136.5, 128.6, 128.4, 128.3, 127.8, 127.2, 125.2, 120.1, 98.2, 71.6, 70.8, 70.6, 70.4, 70.3, 70.1, 69.9, 69.8, 69.3, 67.2, 66.7, 63.1, 59.4, 53.9, 47.4, 40.9, 32.1, 21.80, 21.77, 21.3, 20.9.

HR-ESI-MS (m/z): calcd for C₄₇H₅₉N₅O₁₅Na⁺ (M+Na): 956.3900, found: 958.3858.

### Synthesis of (9H-fluoren-9-yl)methyl N-(2-(2-(2-(245,7-di-acetamido-4,8-di-O-acetyl-1-isopropyl-α-pseudaminosyloxy)ethoxy)ethoxy)ethoxy)ethyl)carbamate (7) (FIG. 10)

To a 25 mL round bottom flask containing 6 (103 mg, 0.108 mmol, 1.0 equiv). Pd/C (10% Pd on activated carbon, 50 mg) and NH₄OAc (30 mg, 0.38 mmol, 4.0 equiv), DCM (2.0 mL) and MeOH (2.0 mL) were added. The mixture was stirred under 1 atm H₂ atmosphere for 1 h, then was filtered through celite to remove catalyst. To this filtrate, NMM (2.0 mL, excess amount) and Ac₂O (1.0 mL, excess amount) was added. After being stirred at r.t. for 2 h, the mixture was concentrated under vacuum. The residue was dissolved in ethyl acetate (50 mL), and the solution was sequentially washed with 1 M HCl (aq) and sat. NaHCO₃ (aq). The organic phase was dried over anhydrous Na₂SO₄, and the solvent was removed under vacuum. The residue was purified by silica gel column chromatography using EtOAc:MeOH 20:1 as eluent. R_{f}(EtOAc:MeOH 20:1)=0.21. The product 7 was obtained as colourless syrup (68.4 mg, 77%).

¹H NMR (500 MHz, CDCl₃): δ−7.76 (d, J=7.5 Hz, 2H, ArH), 7.61 (d, J=7.0 Hz, 2H, ArH), 7.40 (t, J=7.5 Hz, 211, ArH), 7.31 (t, J=7.5 Hz, 2H, ArH), 6.31 (d, J=10.5 Hz, 1H, NH), 6.25 (d, J=10.0 Hz, 1H, NH), 5.70 (t, J=5.0 Hz, 1H, NH), 1.28 (d, J=6.5 Hz, 3H, H-9), 1.29 (d, J=6.0 Hz, 3H, (CH₃)₂CH), 5.24 (dt, J₁=12.0 Hz, J₂=4.5 Hz, 1H, H-4), 5.19 (d, J₁=6.5 Hz, J₂=3.5 Hz, 1H, H-8), 5.06-5.13 (m, 1H, (CH₃)₂CH), 4.48-4.60 (m, 2H₁, H-5, H-7), 4.36-4.43 (m, 2H, C₁₂H₈CHCH₂), 4.31 (d, J=10.5 Hz, 1H, H-6), 4.22 It, J=6.5 Hz, 1H, C₁₂H₈CHCH₂), 3.55-3.76 (m, 13H, OCH₂CH₂O), 3.51 (dt, J=10.5 Hz, J₂=2.5 Hz, 1H, OCH₂CH₂O), 3.36-3.43 (m, 2H, OCH₂CH₂O), 2.12 (dd, J₁=13.0 Hz, J₂=5.0 Hz, 1H, H-3e), 2.03 (s, 3H, CH₃CO), 2.00 (s, 3H, CH₃CO), 1.96 (s, ³H, CH₃CO), 1.91 (s, ³H, CH₃CO), 1.83 (t, J=13.0 Hz, 1H, H-3a), 1.30 (d, J=6.0 Hz, 3H, (CH₃)₂CH).

¹³C NMR (125 MHz, CDCl₃): δ=171.3, 170.9, 170.6, 170.3, 167.2, 156.8, 144.1, 141.4, 127.8, 127.1, 125.2, 120.0, 98.6, 71.0, 70.6, 70.25, 70.17, 70.10, 70.07, 69.8, 67.3, 66.7, 63.4, 50.2, 47.4, 45.7, 41.0, 32.5, 23.3, 23.1, 21.8, 21.4, 21.1, 15.4.

HR-ESI-MS (m/z): calcd for C₄₃H₅₉N₃O₁₅Na⁺ (M+Na): 880.3838, found: 880.3850.

### Synthesis of 3-(1,3-dimethoxy-1,3-dihydroisobenzofuran-5-yl)-N-(2-(2-(2-(2-(5,7-di-acetamido-4,8-di-O-acetyl-1-isopropyl-β-pseudaminosyloxy)ethoxy)ethoxy)ethoxy)ethyl)propanamide (9) (FIG. 11)

To a 10 mL round bottle flask containing 7 (21.8 mg, 0.254 mmol, 1.0 eq) was added 3 mL 20% diethyl amine in MeCN (v/v) solution and the solution was stirred at r.t. for 6 h. The mixture was concentrated under vacuum to remove diethylamine and the residue was redissolved in DCM (4 mL). To the above solution was added acid 8 (32 mg, 5.0 equiv), followed by EDCI (25 mg, 5.0 equiv) and DIPEA (45 μL, 10.0 equiv), and the reaction was monitored by TLC. When completed, the mixture was dilute with ethyl acetate (20 mL) and subsequently washed with NaHCO₃ (aq) and brine. The organic phase was dried over anhydrous Na₂SO₄, and the solvent was removed under vacuum. The residue was purified by silica gel column chromatography using EtOAc:methanol 10:1 as eluent. The product 9 was obtained as white solid (15.5 mg, 71%). Rr (EtOAc:MeOH 10:1)=0.25. The ratio of each isomer cannot be determined using NMR due to extensive overlapping of the chemical shifts.

¹H NMR (400 MHz, CDCl₃): δ=7.15-7.54 (m, 5.8H), 6.99-7.13 (m, 11H), 6.59-6.78 (m, 1H), 6.26 (d, J=3.5 Hz, 0.71H), 5.98-6.03 (m, 111), 5.83-5.92 (m, 1H), 5.59-5.65 (m, 0.7H), 5.23 (qd, J₁=6.5 Hz, J₁=3.0 Hz, 111), 5.03-5.13 (m, 111), 4.73 (d, J=16.5 Hz, 1H), 4.46-4.56 (m, 211), 3.92-4.06 (m, 2.411), 3.75 (t, J=8.5 Hz, 1.211), 3.48-3.68 (m, 14H), 3.34-3.48 (m, 11H), 3.25-3.34 (m, 6H), 2.84-3.06 (m, 6H), 2.70 (s, 2.611), 2.61 (t. J=9.5 Hz, 0.7H), 2.38 (dt, J₁=16.5 Hz, J₂=5.0 Hz, 1H), 2.25-2.33 (m, 0.711), 1.93-2.05 (m, 11H), 1.90 (s, 4H), 1.20-1.34 (m, 19H).

¹³C NMR (100 MHz, CDCl₃): δ=172.3, 172.1, 171.5, 170.58, 170.50, 170.1, 167.3, 143.3, 143.1, 141.3, 139.07, 138.99, 138.90, 138.81, 138.79, 136.71, 136.59, 136.45, 136.34, 136.31, 136.15, 135.92, 135.88, 133.7, 130.40, 130.28, 130.20, 130.06, 128.2, 127.24, 127.00, 126.99, 126.76, 126.68, 126.59, 123.13, 123.10, 122.95, 122.90, 122.87, 122.7, 110.0, 106.52, 106.50, 105.48, 105.44, 105.43, 105.41, 101.2, 99.2, 72.5, 70.35, 70.32, 70.29, 70.27, 70.06, 69.96, 69.94, 69.83, 69.81, 67.8, 63.7, 54.64, 54.54, 54.37, 54.33, 54.28, 54.27, 54.19, 54.12, 54.09, 53.7, 53.6, 53.5, 50.6, 45.9, 45.3, 43.3, 41.5, 39.2, 38.05, 38.04, 37.76, 37.67, 35.7, 35.6, 35.46, 35.44, 34.0, 32.9, 31.90, 31.88, 31.51, 31.45, 30.9, 30.7, 30.56, 30.53, 29.76, 29.67, 29.63, 29.60, 29.50, 29.47, 29.33, 29.29, 29.25, 29.17, 29.10, 27.20, 27.15, 25.3, 24.93, 24.85, 23.2, 22.94, 22.92, 22.7, 21.7, 21.2, 20.9, 14.7, 14.09, 14.03, 14.02.

HR-ESI-MS (m/z): calcd for C₄₁H₆₃N₃O₁₇Na⁺ (M+N⁺): 892.4050, found: 892.4061.

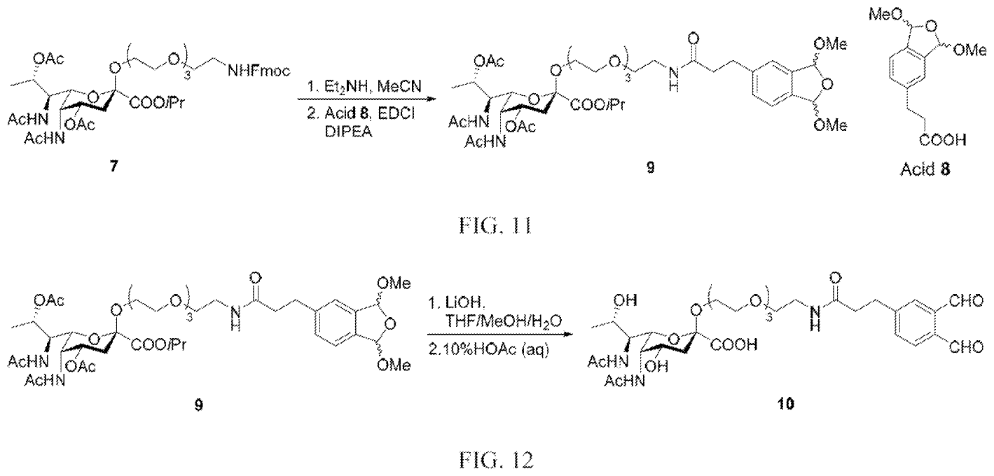

### Synthesis of 3-(3,4-diformylphenyl)-N-(2-(2-(2-(2-(5,7-di-acetamido-α-pseudaminosyloxy)ethoxy)ethoxy)ethoxy)ethyl)propanamide (10) (FIG. 12)

Lithium hydroxide monohydrate (9.4 mg, 0.224 mmol, 10.0 eq) was dissolved in a mixture of THF, MeOH and H₂O (3 mL+0.75 mL+0.75 mL) to obtain the 0.5 mM LiOH solution. To a 10 mL round bottle flask containing compound 9 (19.5 mg, 0.0224 mmol, 1.0 equiv) was added the solution above and the mixture was stirred at r.t for 48 hours. Then the mixture was neutralized by Dowex 50 H⁺ resin and filtered. The filtrate was evaporated under vacuum and the residue was added 3 mL 10% HOAc aqueous solution (v/v) and stirred at r.t for 6 hours. The mixture was then evaporated under vacuum and purified by RP HPLC: t_{R}=21.2 min (Column: Vydac 218TP C18 (300 Å) column (Grace Davison Discovery Sciences); eluent A: MeCN and B: H₂O; gradient: sample was run at 10 ml/min with a gradient of 5-20% A over 35 min; detection: UV 200 nm. The compound 10 was obtained as a mixture of dialdehyde and hemiacetal as white solid (13.2 mg, 77%).

¹H NMR (500 MHz, D₂O): δ=7.22-7.98 (m, 3H), 6.42 (s, 1H), 4.05-4.18 (m, 5H), 3.75 (d, J=10.0 Hz, 1H), 3.31-3.69 (m, 15H), 3.2 (s, 2H), 2.93 (t, J=7.0 Hz, 1H), 2.51 (t, J=7.0 Hz, 1H), 2.01 (d, J=11.0 Hz, 1H), 1.90 (s, 3H), 1.88 (s, 3H), 1.48 (t, J=12.5 Hz, 1H), 1.04 (d, J=6.0 Hz, 3H).

¹³C NMR (125 MHz, D₂O): δ=175.5, 174.8, 174.6, 173.7, 142.8, 139.2, 130.4, 122.8, 117.4, 115.2, 100.4, 70.4, 69.7, 69.5, 69.3, 68.8, 66.9, 64.9, 62.5, 53.6, 48.6, 38.8, 37.3, 35.1, 31.2, 22.04, 21.92, 15.6.

HR-ESI-MS (m/z): calcd for C₃₂H₄₇Na₃O₁₄Na⁺ (M+Na⁺): 720.2950. found: 720.2959.

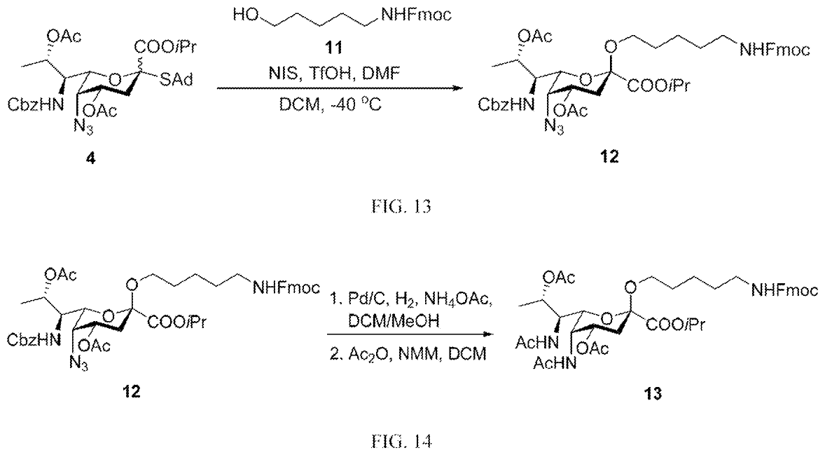

### Synthesis of (9H-fluoren-9-yl)methyl N-(4,8-di-O-acetyl-5-azido-7-N-benzyloxycarbonyl-1-isopropyl-α-pseudaminosyloxy)pentanylcarbamate (12) (FIG. 13)

To a flame-dried Schlenk tube, flame-dried AW-300 molecular sieves (100 mg) were added under argon, followed by Pse donor 4 (41.7 mg, 0.0500 mmol, 1.0 equiv), acceptor 11 (46.0 mg 0.100 mmol, 2.0 equiv), anhydrous DCM (freshly distilled over CaH₂, 1.0 mL), and anhydrous DMF (17.0 μL, 0.250 mmol, 5.0 equiv). After being stirred at r.t. for 1 h. the mixture was cooled to −78° C., and N-iodosuccinimide (27.0 mg, 0.120 mmol, 2.4 equiv) was added. Finally, triflic acid (0.450 μL, 0.1 equiv) was added dropwise to initiate the reaction, and the mixture was stirred at −40° C. for 8 h. Upon full conversion, as indicated by TLC, the reaction was quenched by addition of Et₃N. The mixture was diluted with ethyl acetate and filtered through celite. The organic phase was subsequently washed with sat. NaHCO₃ (aq), dried over anhydrous Na₂SO₄ and concentrated under vacuum. The product 12 was purified by silica gel column chromatography using n-hexane:ethyl acetate 2:1 v/v as eluent. R_{f}(n-hexane:ethyl acetate 1:1)=0.34. Only the a anomer was obtained (J_{Cl-Hz}=0 Hz, measured on Advance DRX Bruker 500 MHz NMR spectrometer by non-decoupled ¹³C spectroscopy) as a white solid (35.5 mg, 83%).

¹H NMR (500 MHz, CDCl₃): δ=7.74 (d, J=10.5 Hz, 21, ArH), 7.54 (d, J=6.7 Hz, 2H, ArH), 7.35-7.41 (m, 3H. ArH), 7.19-7.33 (m, 6H. ArH), 5.77 (d, J=9.6 Hz, 1H, NH), 5.34 (dt, Jr 11.0 Hz, J₂=4.1 Hz, 1H, H-4), 5.21-5.30 (m, 1H, H-8), 4.99-5.12 (m, 3H, PhCH₂O, CH(CH₃)₂), 4.94 (t, J=5.7 Hz, 1H, NH), 4.30-442 (m, 3H, H-7, C₁₂H₈CHCH₂), 4.09-4.13 (m, 1H, C₁₂H₈CHCH₂), 3.98 (s, 1H, H-5), 3.88 (d, J=8.7 Hz, 1H, H-6), 3.48-3.58 (m, 11H), 3.27-3.38 (m, 1H), 3.06-3.22 (m 211), 2.21 (dd, J=12.6 Hz. J₂=4.7 Hz, 1H, H-3e), 2.11 (t, J=12.6 Hz, 1H, H-3a), 2.09 (s, ³H, CH₃CO), 2.00 (s, 3H, CH₃CO), 1.46-1.61 (m, 41H), 1.36 (d, J=6.2 Hz, 3H, H-9), 1.23-1.27 (m, 8H). ¹³C NMR (125 MHz, CDCl₃): δ=170.3, 170.0, 166.4, 156.7, 143.95, 143.91, 141.29, 141.27, 136.3, 128.5, 128.44, 128.08, 127.6, 127.0, 124.93, 124.85, 119.9, 71.78, 71.69, 69.85, 69.70, 69.62, 69.39, 69.30, 66.96, 66.57, 63.18, 59.23, 59.13, 59.9, 47.17, 47.09, 41.1, 32.1, 29.7, 28.8, 23.4, 21.67, 21.63, 21.58, 20.7.

HR-ESI-MS (m/z): calcd for C₄₄H₅₃N₅O₁₂Na⁺ (M+Na⁺): 866.3583, found: 866.3588.

### Synthesis of (9H-fluoren-9-yl)methyl N-(5,7-di-acetamido-4,8-di-O-acetyl-1-isopropyl-α-pseudaminosyloxy)pentanylcarbamate (13) (FIG. 14)

To a 25 mL round bottom flask containing 12 (79.2 mg, 0.094 mmol, 1.0 equiv), Pd/C (10% Pd on activated carbon, 50 mg) and NH₄OAc (29.2 mg, 0.38 mmol, 4.0 equiv), DCM (2.0 mL) and MeOH (2.0 mL) were added. The mixture was stirred under H₂ atmosphere (1 atm) for 1 h. then was filtered through celite to remove catalyst. To this filtrate, NMM (2.0 mL, excess amount) and Ac₂O (1.0 mL, excess amount) were added. After being stirred at r.t. for 2 h, the mixture was concentrated under vacuum. The residue was dissolved in ethyl acetate (50 mL), and the solution was sequentially washed with 1 M HCl (aq) and sat. NaHCO₃ (aq). The organic phase was dried over anhydrous Na₂SO₄, and the solvent was removed under vacuum. The residue was purified by silica gel column chromatography using EtOAc:MeOH 20:1 as eluent. R_{f}(EtOAc:MeOH 20:1)=0.33. The product 13 was obtained as white solid (55.4 mg, 77%). ¹H NMR (500 MHz, CDCl₃) δ 7.76 (d, J=7.5 Hz, 211, ArH), 7.67 (d, J=7.5 Hz, 1H, ArH), 7.63 (d, J=7.5 Hz, 1H, ArH), 7.40 (t, J=7.5 Hz, 2H, ArH), 7.36-7.28 (m, 21H, ArH), 6.22 (d, J=9.0 Hz, 1H, NH), 6.20 (d, J=8.6 Hz, 1, NH), 5.23 (dt, J₁=12.2 Hz, J₂=4.2 Hz, 1H, H-4), 5.16 (qd, J₁=6.5 Hz, J₂=2.8 Hz, 1H, H-8), 5.13-5.07 (m, 1H, CH(CH₃)₃), 5.03 (t. J=5.8 Hz, 1H, NH), 4.57 (d, J=9.1 Hz, 1H, H-5), 4.55-4.47 (m, 2H, H-7, C₁₂H₈CHCH₂), 4.31-4.22 (m, 2H, C₁₂H₈CHCH₂), 4.04 (d, J=10.4 Hz, 1H, H-6), 3.54-3.46 (m, 1H. OCH₂), 3.41-3.34 (m, 1H, OCH₂), 3.29-3.10 (m, 2H, NHCH₂), 2.16 (dd, J₃=13.0 Hz, J₂=4.7 Hz, 11H, H-3e), 2.04 (s, 3H, CH₃CO), 2.00 (s, ³H, H₃CO), 1.97 (s, 3H. CH₃CO), 1.85 (s, ³H, CH₃CO), 1.80 (t, J=12.8 Hz, 1H, H-3a), 1.68-1.38 (m, 6H, (CH₂)₃), 1.33-1.25 (m, 911, H-9, CH(CH)₂). ¹¹C NMR (125 MHz, CDCl₃) δ 171.03, 170.61, 170.53, 169.94, 167.11, 156.77, 144.23, 143.70, 141.32, 141.23, 127.67, 127.63, 127.17, 126.95, 125.20, 125.13, 119.93, 119.89, 98.75, 71.30, 70.21, 70.03, 67.21, 66.90, 63.87, 53.40, 50.38, 47.08, 45.63, 41.07, 32.52, 29.98, 28.87, 23.88, 23.21, 23.15, 21.66, 21.65, 21.30, 20.96, 15.79.

HR-ESI-MS (m/z): calcd for C₄₀H₅N₃O₁₂Na (M+Na⁺): 790.3521, found: 790.3527.

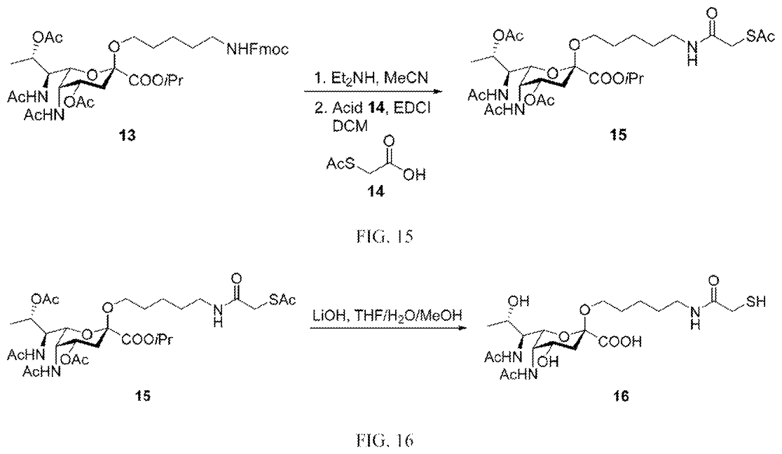

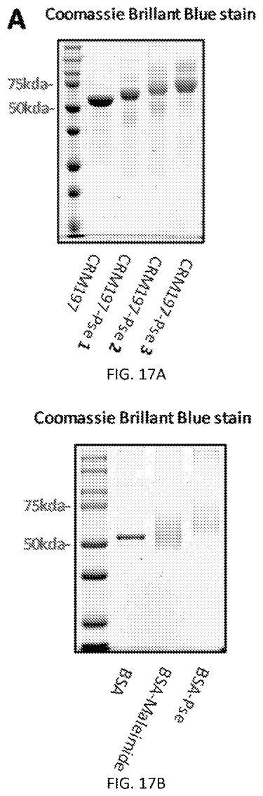

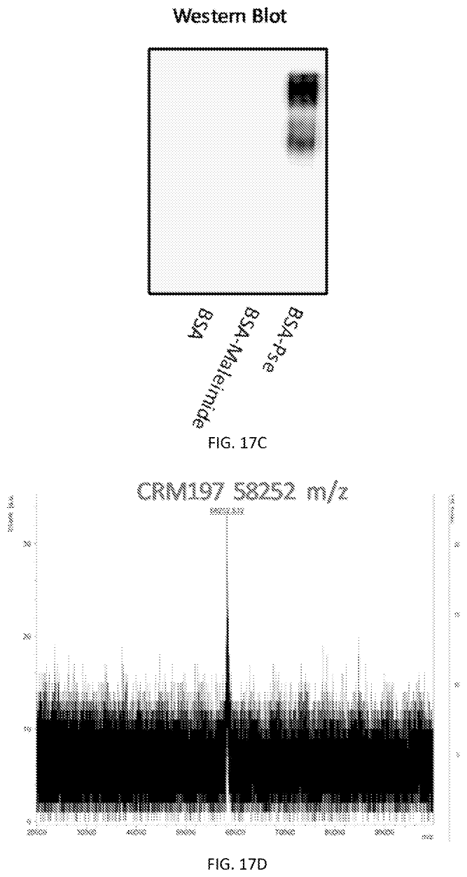

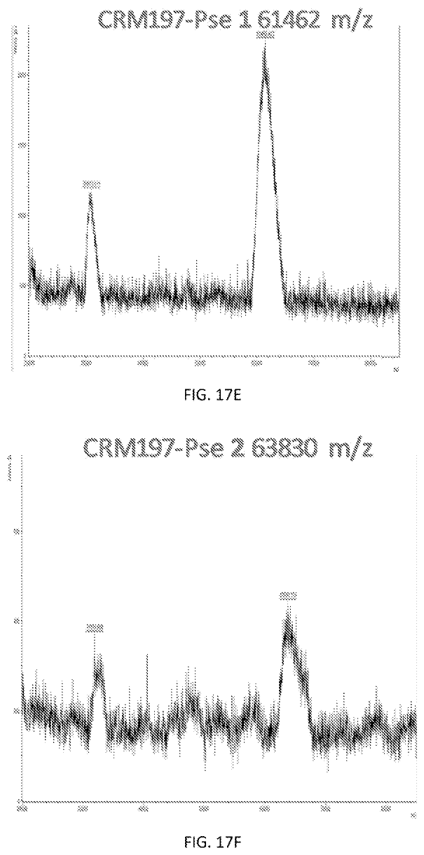

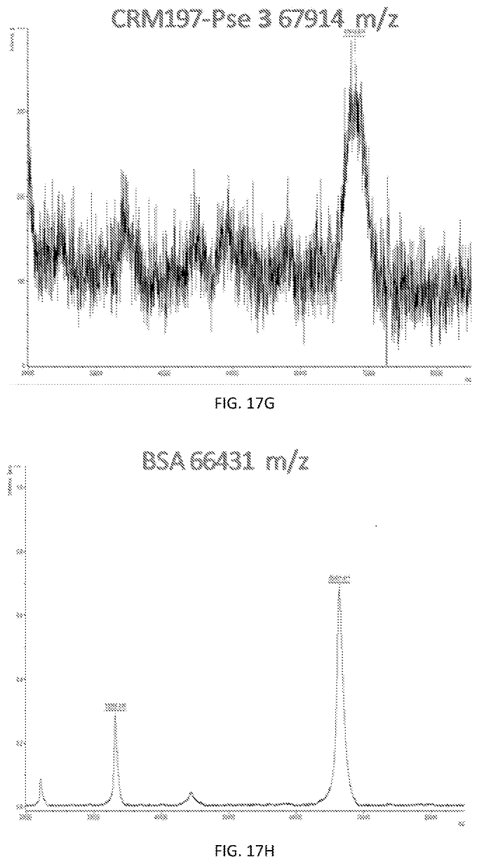

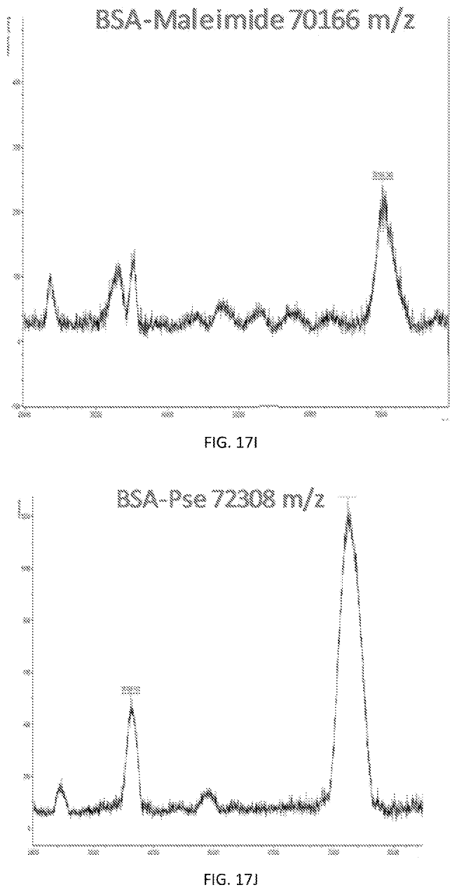

### Synthesis of S-(2-((5-(5,7-di-acetamido-4,8-di-O-acetyl-1-isopropyl-α-pseudaminosyloxy) pentanyl)amino)-2-oxoethyl) ethanethioate (15) (FIG. 15)

To a 10 mL round bottle flask containing 13 (27.7 mg, 0.361 mmol, 1.0 equiv) was added 3 mL 20% diethyl amine in MeCN (v/v) solution and the solution was stirred at r.t. for 6 h. The mixture was concentrated under vacuum to remove diethyl amine and the residue was redissolved in DCM 4 mL. To the solution above was added acid 14 (14.5 mg, 3.0 equiv), followed by EDCI (20.7 mg, 3.0 eq) and monitored by TLC. When completed, the mixture was dilute with ethyl acetate 20 mL and subsequently washed with NaHCO₃ (aq) and brine. The organic phase was dried over anhydrous Na₂SO₄, and the solvent was removed under vacuum. The residue was purified by silica gel column chromatography using EtOAc:methanol 10:1 as eluent. R_{f}(EtOAc:MeOH 10:1)=0.25. The product 15 was obtained as white solid (17.4 mg, 73%).

¹H NMR (400 MHz, CDCl₃) δ 7.22 (d, J=10.0 Hz, 11H. NH), 6.69 (d, J=9.4 Hz, 1H, NH), 6.52 (t, J=6.2 Hz, 1H, NH), 5.24-5.15 (m, 211.1H-4, H-8), 5.15-5.04 (m, 11H, CH(CH)₂), 4.56 (td, J₁=10.4 Hz, J₂=3.2 Hz, 1H, H-7), 4.52 (d, J=9.4 Hz, 1H, H-5), 4.02 (dd, J₁=10.6 Hz, J₂=1.9 Hz, 1H, H-6), 3.68 (d, J=15.0 Hz, 1H, CH₂SCOCH₃), 3.54 (d, J=15.0 Hz, 1H. CH₂SCOCH₃), 3.51-3.43 (m, 1H, OCH₂), 3.43-3.34 (m, 2H, 1×OCH₂, 1×NHCH₂), 3.17-3.06 (m, 1H, NHCH₂), 2.42 (s, 3H, CH₃SCOCH₃), 2.13 (dd. J₁=13.1 Hz, J=4.4 Hz, 1H, H-3e), 2.05 (s, 3H, CH₃CO), 2.02 (s, 3H, CH₃CO), 1.99 (s, 3H, CH₃CO), 1.97 (s, 3H. CH₃CO), 1.78 (t J=12.8 Hz, 1H, H-3a), 1.67-1.41 (m, 6H. (CH₂)₃), 1.35-1.26 (m, 9H, H-9. CH(CH₃)₂).

¹³C NMR (100 MHz, CDCl₃) δ=196.03, 172.24, 171.25, 170.88, 170.44, 169.15, 167.18, 98.64, 70.79, 70.17, 70.15, 66.87, 63.59, 50.30, 45.76, 40.16, 33.23, 32.58, 30.27, 29.62, 28.63, 24.45, 22.80, 22.78, 21.63, 21.60, 21.3, 1, 20.88, 15.47.

HR-ESI-MS (m/z): calcd for C₂₉H₄₇N₃O₁₂Na⁺ (M+Na⁺): 684.2773, found: 684.2777.

### Synthesis of 2-mercapto-N-(5-(5,7-di-acetamido-α-pseudaminosyloxy)pentyl)acetamide (16) (FIG. 16)

Lithium hydroxide monohydrate (11.0 mg, 0.263 mmol, 10.0 eq) was dissolved in a mixture of THF, MeOH and H₂O (3 mL+0.75 mL.+0.75 mL) to obtain the 0.5 mM LiOH solution. To a 10 mL round bottle flask containing compound S5 (17.4 mg, 0.0263 mmol, 1.0 equiv) was added the solution above and the mixture was stirred at r.t for 48 h. Then the mixture was neutralized by Dowex 50 H⁺ resin and filtered. The filtrate was evaporated under vacuum and the aqueous solution was purified by RP HPLC: t_{R}=9.5 min (Column: Vydac 2181 C18 (300 Å) column (Grace Davison Discovery Sciences); eluent A: MeCN and B H₂O; gradient: sample was run at 10 mL/min with a gradient of 5-35% A over 35 min; detection: UV 200 nm. The compound S6 was obtained as a mixture of thiol and disulfide (9.4 mg, 72%).

¹H NMR (400 MHz, D₂O) δ=4.02-4.16 (m, 4H), 3.70 (d, J=10.1 Hz, 1H, 3.23 (t, J=6.2 Hz, 2H), 3.06-3.16 (m, 3H), 1.94-2.02 (m, 111), 1.88 (s, ³H), 1.87 (s, 3H), 1.38-1.59 (m, 5H), 1.20-1.36 (m, 2H), 1.03 (d, J=6.5 Hz, 3H).

¹³C NMR (100 MHz, D₂O) δ=175.0, 174.6, 1737, 173.5, 163.1, 162.8, 120.6, 117.7, 114.8, 111.9, 100.1, 70.4, 68.6, 66.9, 65.0, 63.6, 53.6, 48.7, 39.5, 35.3, 28.4, 28.1, 27.2, 22.7, 22.0, 21.9, 15.6.

HR-ESI-MS (m/z): calcd for C₂₀H₃₅N₃O₉SNa⁺ (M+Na⁺): 516.1986. found: 516.1991.

### Preparation of CRM197-Pse Conjugate 1-3

Ortho-Phthalaldehyde (OPA) modified pseudaminic acid species 10 (20.0, 30.0 or 50.0 equiv) was dissolved in 0.1 M Phosphate Buffered Saline (PBS buffer), pH 7.4 (200 μL) and added to a solution of lyophilized CRM197 (1.0 mg, 17.1 nmol, 1.0 equiv) in 0.1 M PBS buffer, pH 7.4 (1.5 mL). The mixture was incubated at room temperature for 6 h, then diluted with sterile water to 5 mL and dialyzed using a centrifugal filter (10 kDa MWCO, Milipore, Amicon Ultra) at 4° C. The protein solution was concentrated to 500 μL and diluted with sterile water to 5 mL. The process was repeated three times and finally concentrated to 300 μL, 20 μL was taken for analysis and the protein solution was rebuffered with 0.1 M PBS buffer, pH 7.4 to 5 mL, concentrated to 500 μL and stored at −40° C. prior to immunization. CRM197-Pse 1, 2 and 3 refer to the conjugation with 20, 30 and 50 equivalents of Pse respectively.

### Preparation of Pse-BSA Conjugate 17

A solution of N-(s-maleimidocaproxy)sulfosuccimide ester (sulfo-EMCS) (2.1 mg, 5.2 μmol) in 0.1 M PBS buffer, pH 7.4 (200 μL) was added to a stirred solution of bovine serum albumin (BSA, 3 mg, 51.7 nmol) in 0.1 N PBS buffer pH 7.4 (1.5 mL) at room temperature. The mixture was stirred for 2 h, then diluted with sterile water to 5 mL, and dialyzed using a centrifugal filter (10 kDa MWCO, Milipore, Amicon Ultra) at 4° C. The process was repeated three times and finally concentrated to 300 μL, 20 μL were taken for analysis, and the protein solution was re-buffered to 0.1 M PBS buffer pH 7.4 in 5 mL and used in the next step.

Pse-thiol species 16 (2.6 mg, 5.2 μmol resp, to the monomer, the ratio of thiol and disulfide was estimated by UPLC) in 0.1 M PBS buffer pH 7.4 (0.2 mL) was treated at room temperature with tris(2-carboxyethyl)phosphine (TCEP, 25 μL of a 100 mM stock solution, pH 7.4), left for 1 h at that temperature under an argon atmosphere and added to the solution of the activated protein. The mixture was stirred at room temperature for 16 h. The glycoconjugate was then treated at room temperature with L-cysteine (625 μg, 5.1 μmol) in 100 μL sterile water and left for 1 h. After that, the mixture was diluted with sterile water to 5 mL and dialyzed using a centrifugal filter (10 kDa MWCO, Milipore. Amicon Ultra) at 4° C.. The process was repeated three times and finally concentrated to 300 μL, 20 μL were taken for analysis, and the protein solution was re-buffered to 0.1 M PBS buffer pH 7.4 in 5 mL, concentrated to 500 μL and stored at −40° C.

### Characterization of Synthetic Pse-CRM197 Conjugates 1-3 and Pse-BSA Conjugate 17

The conjugate was prepared in 1× SDS-PAGE sample loading dye and was resolved on 10% SDS-PAGE. The electrophoresis was carried out at 80 V for 30 min and 160V for 30 min in electrode buffer and gel was stained by Coomassie Brilliant Blue. The average molecular size of the glycoconjugate was determined by Matrix-assisted laser desorption/ionization (MALDI) analysis on Bruker ultrafleXtreme mass spectrometer using 2,5-dihydroxybenzoic acid (DHB) as matrix. Recombinant CRM197 protein (Pfenex Inc, San Diego), and BSA (Sangon Biotech, Shanghai) was used as standard.

TABLE 1
Antigen loading calculation of CRM197-Pse conjugate.
MW of saccharide is 334 (697 with spacer)

| | | Average sugar/ | Antigen load |
|---|---|---|---|
| | MW of conjugate | protein ratio | (glycan, w/w): |
| CRM197-Pse 1 | 61462 | 4.76 | 2.56% |
| CRM197-Pse 2 | 63830 | 8.27 | 4.28% |
| CRM197-Pse 3 | 67914 | 14.34 | 6.97% |

### Example 2-Antibody Responses of Pre-CRM197 Vaccines

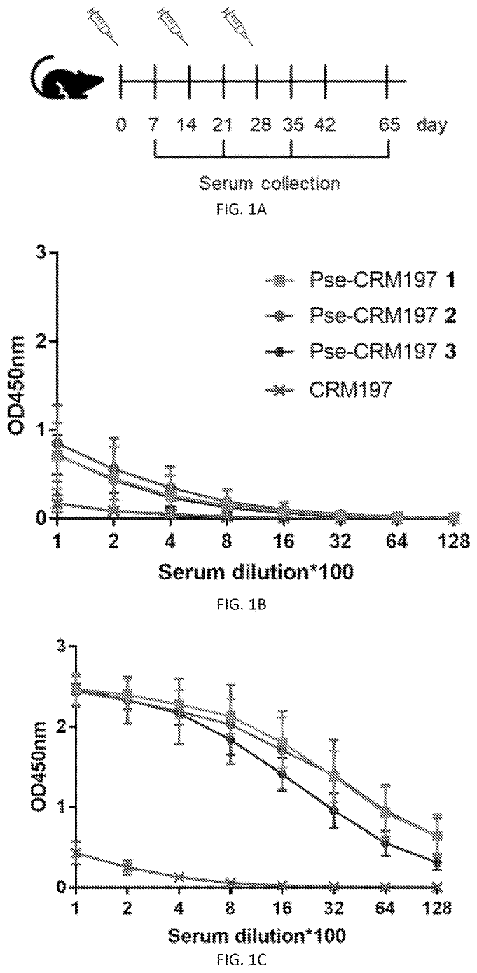

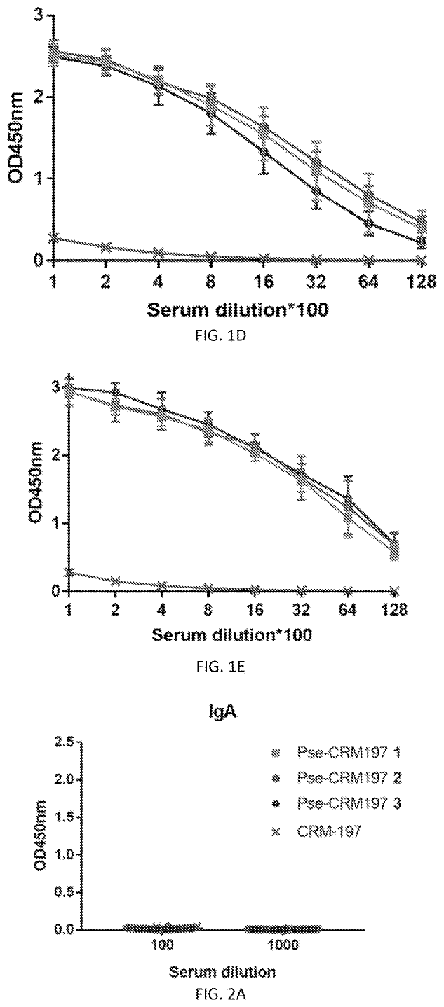

The immunogenicity of Pse-CRM197 conjugates was assessed by immunizing male C57BL163 mice mixed with aluminum hydroxide in a prime boost regimen (FIG. 1A). The control group received CRM197 mixed with aluminum hydroxide in PBS. The Pse specific antibody response in post-immune sera was characterized by ELISA. Mice that received the first dose of all the three Pse vaccines produced a bit antibody response to BSA-Pse, while no humoral immune response was observed in the CRM197 control mice (FIG. 1B). The antibody titers increased significantly on day 21, one week after receiving the second dose for all the three Pse-CRM197 vaccines (FIG. 1C). The antibody response of Pse-CRM197 3 was found to be a little lower compared to that of Pse-CRM197 1 and Pse-CRM197 2, which might be due to the over-crowded sugar content. On day 35, one week after receiving the third dose, the antibody titers for all the three Pse vaccines maintained stable (FIG. 1D). Furthermore, the antibody responses sustained for all the three Pse vaccines in one month, namely five weeks after the last immunization (FIG. 1E). And the titers for Pse-CRM197 3 caught up to levels of Pse-CRM197 1 and Pse-CRM197 2 finally. These data demonstrated that vaccination of the Pse vaccines elicited significant levels of IgG against Pse-BSA, whereas control mice had no detectable antigen-specific IgG.

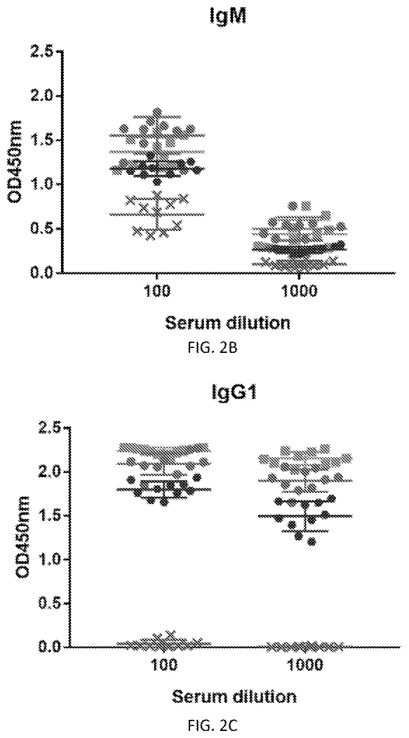

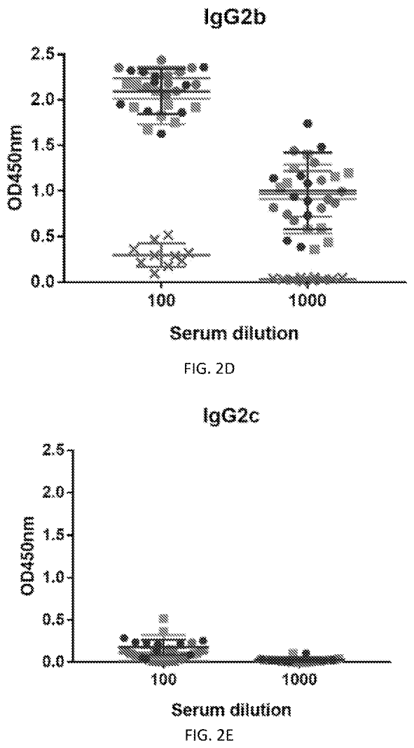

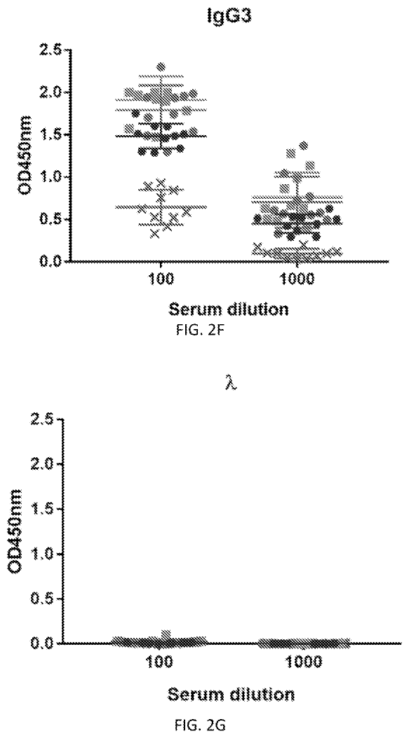

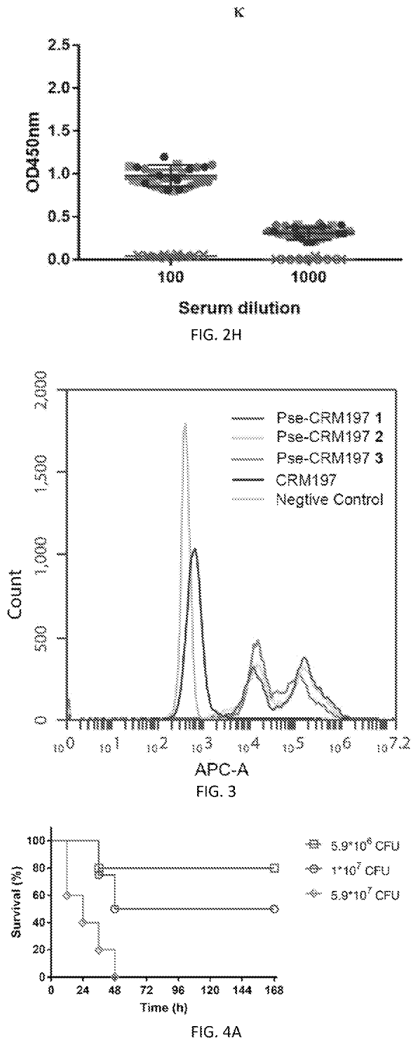

To identify the immune responses during immunization of the Pse-CRM197 vaccines, isotyping of Pse specific antibodies in post-immune sera on day 35 was determined by ELISA. None of the formulations induced the production of detectable amounts of IgA antibodies (FIG. 2A). For the three Pse-CRM197 vaccines, IgG1, IgG2b, and IgG3 contributed the bulk of the Pse-specific IgG titer, while a weak IgG2c response was observed, demonstrating that immunization with the Pse-CRM197 vaccines produced antibodies of three subtypes (FIGS. 2C-2F). In contrast, mice vaccinated with CRM197 failed to elicit Pse-specific IgG All the detected Pse specific antibodies were with kappa light chains. These data indicated that vaccination of all the three Pse vaccines elicited humoral immune responses and produced sustained and significant levels of Pse-specific antibodies of IgG type.

### Example 3—Flow Cytometry Analysis of Post-Immune Sera Toward A. baumannii Strain Ab2

The binding capacity of post-immune sera toward Pse producing A. baumannii strain Ab2 was further determined by flow cytometry analysis. Bacteria incubated with the three Pse vaccines treated post-immune sera showed similar profiles, while the CRM-197 control resembled the negative control (FIG. 3). The significant difference of fluorescent strength illustrated that glycoconjugate-boosted sera can recognize bacteria bearing Pse on the surface.

### Example 4-Immunization with Pse-CRM197 Vaccines Protects Mice from A. baumannii Infection

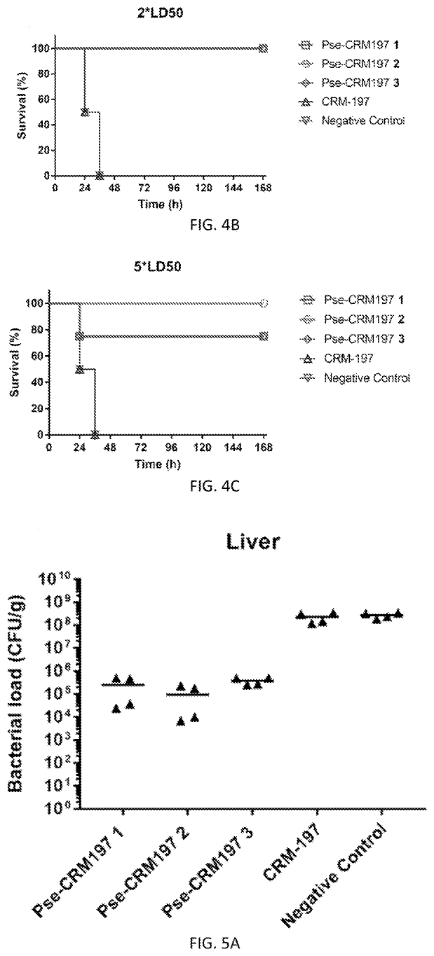

To characterize the efficacy of the Pse vaccines, the vaccinated and control mice were challenged with A. baumannii strain Ab2 using a mouse sepsis model. The LD50 of strain Ab2 was determined by infecting mice with different doses of bacteria. Infection of mice with 5.9×10⁶, 1×10⁷ and 5.9×10⁷ CFU of strain Ab191 led to 20%, 50% and 100% mortality, respectively (FIG. 4A). It was next determined if the response produced by immunization with the Pse vaccines was sufficient to provide protection from infection with A. baumannii. Mice immunized as previously described, at week 0, 2 and 4, were challenged with A. baumannii strain Ab2 two weeks after the final immunization and monitored for survival over 7 days (n=4 mice/group). Vaccinated mice challenged with 2.0-10⁷ CFU (2×LD50) of A. baumannii strain Ab2 were completely protected from challenge, whereas all control mice received CRM-197 and negative control mice died within 36 h (FIG. 4B). When challenged with 5.0×10⁷ CFU (5×LD50) of strain Ab2, 25% mortality was recorded for mice received Pse-CRM197 1 and Pse-CRM197 3, 0% mortality for mice received Pse-CRM197 2, 100% mortality for control mice received CRM-197 as well as the negative control mice (FIG. 4C). These data indicated that these Pse vaccines all provided protection from infections caused by Pse-bearing A. baumannii strains.

### Example 5—Immunization with Pse-CRM197 Vaccines Reduce Post-Infection Bacterial Loads

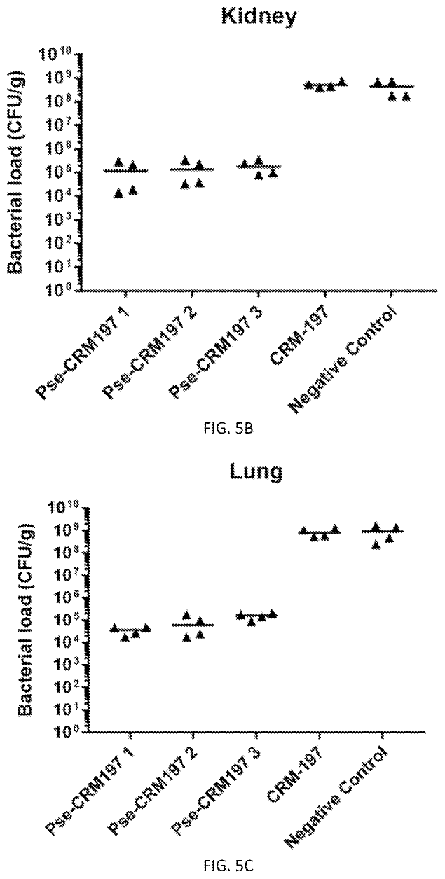

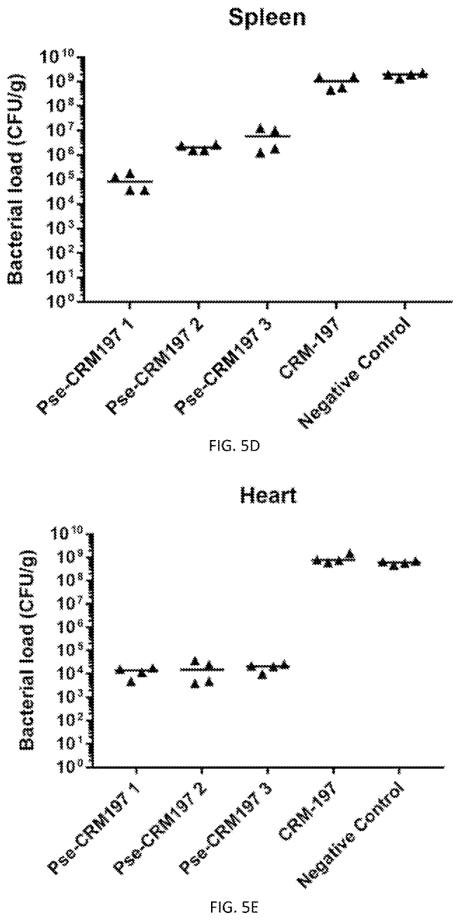

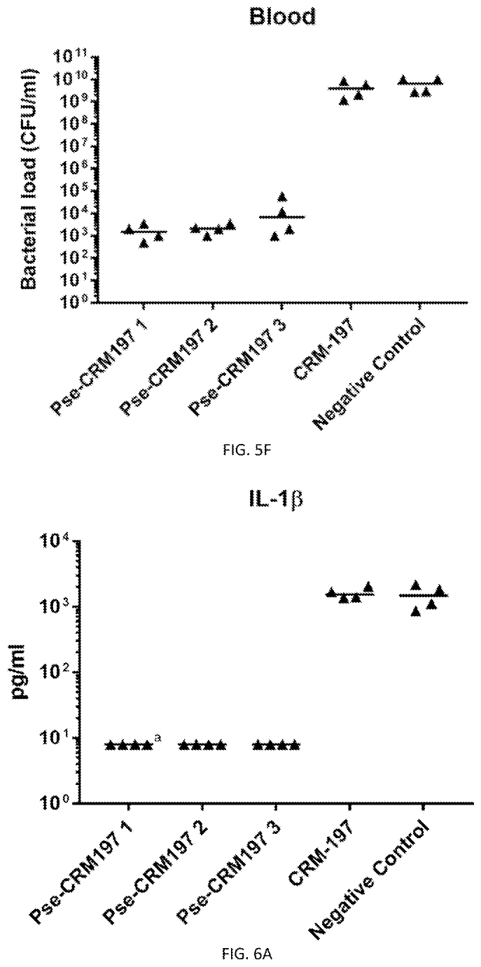

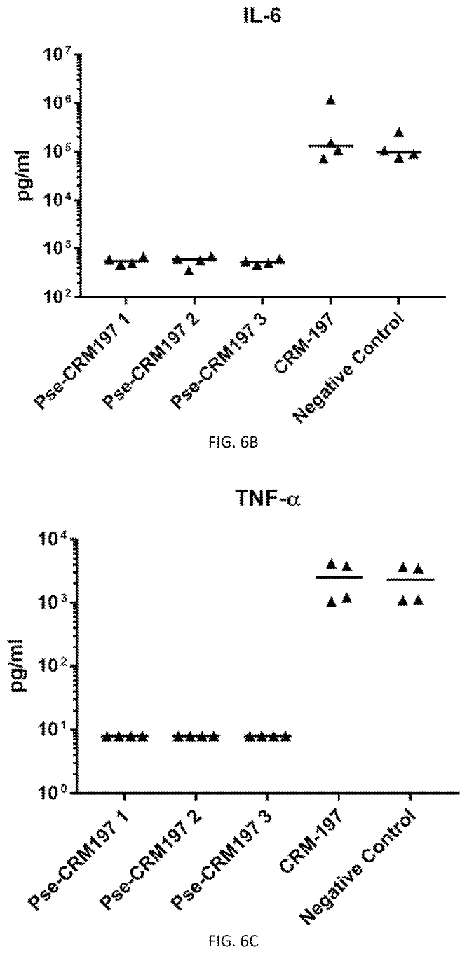

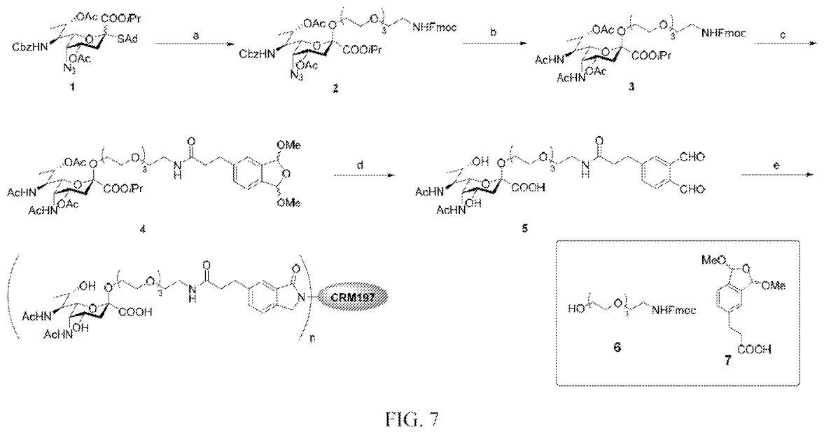

Using the A. baumannii sepsis model, the effect of vaccination on post infection tissue bacterial loads was determined by measuring the quantities of viable bacteria in blood and tissues of vaccinated and control mice (n=4 mice/group) 12 h after infection with 5.0×10⁷ CFU (5×LD50) of strain Ab2. Vaccination resulted in a reduction in tissue bacterial loads of 10³˜10⁵-fold compared to those in control mice for all tissues tested, and 10⁶-fold for blood (FIGS. 5A-5F). Next serum levels of proinflammatory cytokines IL-1β, IL-6, and TNF-α were measured to determine if immunization with these Pse-CRM197 vaccines was able to protect infected mice from release of these cytokines which were produced during bacterial sepsis. It was found that levels of all three cytokines were significantly lower in vaccinated mice than in control mice, suggesting that vaccinated mice did not experience the proinflammatory cytokines release associated with the development of septic shock (FIGS. 6A-6C).

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Howard, A.; O'Donoghue, M.; Feeney, A.; Sleator. R. D., Acinetobacter baumannii: an emerging opportunistic pathogen. Virulence 2012, 3 (3), 243-250.
2. Morris, F. C.; Dexter, C.; Kostoulias, X.; Uddin, M. L; Peleg, A. Y., The Mechanisms of Disease Caused by Acinetobacter baumannii. Front Microbiol 2019, 10, 1601.
3. Antunes, L. C. S.; Visca, P.; Towner, K. J., Acinetobacter baumannii: evolution of a global pathogen. Pathogens and Disease 2014, 71 (3), 292-301.
4. Butler, D. A.; Biagi. M.; Tan, X.; Qasmieh, S.; Bulman. Z. P.; Wenzler. E., Multidrug Resistant Acinetobacter baumannii: Resistance by Any Other Name Would Still be Hard to Treat. Curr Infect Dis Rep 2019, 21 (12), 46.
5. Dijkshoorn, L.; Nemec, A.; Seifert. H., An increasing threat in hospitals: multidrug-resistant Acinetobacter baumannii, Nat Rev Microbiol 2007, 5 (12), 939-51.
6. Poirel, L.; Nordmann, P., Carbapenem resistance in Acinetobacter baumannii: mechanisms and epidemiology. Clin Microbiol Infect 2006, 12 (9), 826-36.
7. Qureshi, Z. A.; Hittle, L. E.: O'Hara, J. A.; Rivera, J. I.; Syed, A.; Shields, R. K.; Pasculle, A. W.; Ernst, R. K.; Doi. Y., Colistin-resistant Acinetobacter baumannii: beyond carbapenem resistance. Clin Infect Dis 2015, 60 (9), 1295-303.
8. He, T.; Wang. R.; Liu, D.; Walsh. T. R.; Zhang, R.; Lv, Y.; Ke, Y.; Ji, Q.; Wei, R.; Liu, Z.; Shen, Y.; Wang, G.; Sun, L.; Lei, L.; Lv, Z.; Li, Y.; Pang, M.; Wang, L.; Sun, Q.; Fu. Y.; Song, H.; Hao. Y.; Shen. Z.; Wang. S.; Chen. G.; Wu, C.; Shen, J.; Wang, Y., Emergence of plasmid-mediated high-level tigecycline resistance genes in animals and humans. Nat Microbiol 2019, 4 (9), 1450-1456.
9. Global Priority List of Antibiotic-resistant Bacteria to Guide Research. Discovery, and Development of New Antibiotics. The World Health Organization: 2017,
10. Zeng, P.; Yi, L.; Xu, J.; Gao, W.; Xu, C.; Chen, S.; Chan. K. F.; Wong, K. Y., Investigation of antibiofilm activity, antibacterial activity, and mechanistic studies of an amphiphilic peptide against Acinetobacter baumannii. Biochim Biophys Acta Biomember 2021, 1863 (6), 183600.
11. Garcia-Quintanilla. M.; Pulido, M. R.; Lopez-Rojas, R.; Pachon, J.; McConnell, M. J., Emerging therapies for multidrug resistant Acinetobacter baumannii. Trends Microbiol 2013, 21 (3), 157-63.
12. Gellings, P. S.; Wilkins, A. A.: Morici. L. A., Recent Advances in the Pursuit of an Effective Acinetobacter baumannii Vaccine. Pathogens 2020, 9 (12), 1066.
13. Micoli, F.; Costantino, P.; Adamo, R., Potential targets for next generation antimicrobial glycoconjugate vaccines. FEMS Microbiol Rev 2018, 42 (3), 388-423.
14. Vella, M.; Pace, D., Glycoconjugate vaccines: an update. Expert Opinion on Biological Therapy 2015, 15 (4), 529-546.
15. Luo, F. J.; Shi, N. M.; Li, L.; Zhang. Z.; Ai. X.; Yang. L. Q.; Bai. Y. H.; Wang, Z. Y.; Lu, Q., Safety observation study on Haemophilus influenzae type B conjugate vaccines injected at different sites in Chinese infants. Biomed Environ Sci 2013, 26 (8), 693-6.
16. Vila-Corcoles, A.; Ochoa-Gondar, O., Preventing pneumococcal disease in the elderly: recent advances in vaccines and implications for clinical practice. Drugs Aging 2013, 30 (5), 263-76.
17. Dretler, A. W.; Rouphael, N. G.; Stephens, D. S., Progress toward the global control of Neisseria meningitidis: 21st century vaccines, current guidelines, and challenges for future vaccine development. Hum Vaccin Immunother 2018, 14 (5), 1146-1160.
18. Jin, C.; Gibani, M. M.; Moore, M.; Juel, H. B.; Jones. E.; Meiring, J.; Harris, V.; Gardner, J.; Nebykova, A.; Kerridge, S. A.; Hill, J.; Thomaides-Brears, H.; Blohmke, C. J.; Yu, L. M.; Angus. B.; Pollard, A. J., Efficacy and immunogenicity of a Vi-tetanus toxoid conjugate vaccine in the prevention of typhoid fever using a controlled human infection model of Salmonella Typhi: a randomised controlled, phase 2b trial. Lancet 2017, 390 (10111), 2472-2480.
19. Tomczyk, S.; Bennett, N. M.; Stoecker, C.; Gierke, R.; Moore, M. R.; Whitney, C. G.; Hadler, S.; Pilishvili. T., Use of 13-valent pneumococcal conjugate vaccine and 23-valent pneumococcal polysaccharide vaccine among adults aged ≥65 years: recommendations of the Advisory Committee on Immunization Practices (ACIP). MMWR Morb Mortal Wkly Rep 2014, 63 (37), 822-5.
20. Torado, G.; Toledo, M. E.; Baly, A.; Fernandez-Santana. V.; Rodriguez, F.; Alvarez, Y.; Serrano, T.; Musachio, A.; Hernandez, L: Hardy, E.; Rodriguez, A.; Hernandez, H.; Aguilar. A.; Sanchez, R.; Diaz, M.; Muzio, V.; Dfana, J.; Rodriguez, M. C.; Heynngnezz. L.; Verez-Bencomo, V., Phase I clinical evaluation of a synthetic oligosaccharide-protein conjugate vaccine against Haemophilus influenzae type b in human adult volunteers. Clin Vaccine Imnunol 2006, 13 (9), 1052-1056.
21. Mettu, R.; Chen, C. Y.: Wu, C. Y., Synthetic carbohydrate-based vaccines: challenges and opportunities. J Biomed Sci 2020, 27(1), 9.
22. Schirm, M.; Soo. E. C.; Aubry. A. J.; Austin, J.; Thibault, P.; Logan, S. M., Structural, genetic and functional characterization of the flagellin glycosylation process in Helicobacter pylori. Mol Microbiol 2003, 48 (6), 1579-92.
23. Knirel, Y. A.; Vinogradov, E. V.: L'Vov V, L.; Kocharova, N. A.; Shashkov, A. S.: Dmitriev, B. A.: Kochetkov, N. K., Sialic acids of a new type from the lipopolysaccharides of Pseudomonas aeruginosa and Shigella boydii. Carbohydr Res 1984, 133 (2), C5-8.
24. Logan, S. M., Flagellar glycosylation—a new component of the motility repertoire?Microbiology (Reading) 2006, 152 (Pt 5), 1249-62.
25. Senchenkova, S. N.; Popova, A. V.; Shashkov, A. S.; Shneider, M. M.; Mei, Z.; Arbatsky. N. P.; Liu, B.; Miroshnikov. K. A.; Volozhantsev, N. V.; Knirel, Y. A., Structure of a new pseudaminic acid-containing capsular polysaccharide of Acinetobacter baumannii LUH5550 having the KL42 capsule biosynthesis locus. Carbohydr Res 2015, 407, 154-7.
26. Vinogradov, E.; Wilde, C.; Anderson. E. M.; Nakhamchik, A., Lam, J. S.; Rowe-Magnus, D. A., Structure of the lipopolysaccharide core of Vibrio vulnificus type strain 27562. Carbohydr Res 2009, 344 (4), 484-90.
27. Goon, S.; Kelly, J. F.; Logan. S. M.; Ewing, C. P.; Guerry. P., Pseudaminic acid, the major modification on Campylobacter flagellin, is synthesized via the Cj1293 gene. Mol Microbiol 2003, 50 (2), 659-71.
28. Lee, I. M.; Yang. F. L.; Chen, T. L.; Liao, K. S.; Ren, C. T.; Lin. N. T.; Chang. Y. P.; Wu, C. Y.; Wu, S. H., Pseudaminic Acid on Exopolysaccharide of Acinetobacter baumannii Plays a Critical Role in Phage-Assisted Preparation of Glycoconjugate Vaccine with High Antigenicity. J. Am Chem Soc 2018, 140 (28)., 8639-8643.
29. Andolina, G.; Wei, R.; Liu, H.; Zhang, Q.; Yang, X.; Cao. H.; Chen. S.; Yan, A.; Li, X. D.; Li. X., Metabolic Labeling of Pseudaminic Acid-Containing Glycans on Bacterial Surfaces. ACS Chemical Biology 2018, 13 (10), 3030-3037.
30. Liu, H.; Zhang, Y.; Wei, R.; Andolina. G.; Li, X., Total Synthesis of Pseudomonas aeruginosa 1244 Pilin Glycan via de Novo Synthesis of Pseudaminic Acid. J Am Chem Soc 2017, 139 (38), 13420-13428,
31. Wei, R.; Liu. H.; Tang. A. H.; Payne, R. J.; Li., X., A Solution to Chemical Pseudaminylation via a Bimodal Glycosyl Donor for Highly Stereocontrolled α- and β-Glycosylation. Org Lett 2019, 21(10), 3584-3588.
32. McConnell, M. J.; Actis, L.; Pachón, J., Acinetobacter baumannii: human infections, factors contributing to pathogenesis and animal models. FEMS Microbiol Rev 2013, 37 (2), 130-155.

1. A compound of formula (I), comprising at least one pseudaminic acid (Pse) moiety conjugated to a carrier protein, wherein the pseudaminic acid moiety is linked to the carrier protein by a linker and a connector:

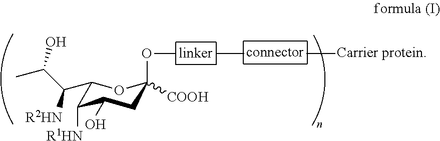

2. The compound of claim 1, wherein the carrier protein is CRM197, diphtheria toxoid (DT), tetanus toxoid (TI), meningococcal outer membrane protein complex (OMPC), H. influenzae protein D (HiD), keyhole limpet hemocyanin (KLH), bovine serum albumin (BSA), or human serum albumin (HSA).

3. The compound of claim 1, wherein about 5 to about 20 Pse compounds are conjugated to the carrier protein, wherein n is about 4 to about 20.

4. The compound of claim 1, wherein the R¹ group on the N5 position is acetyl, formyl, or (R)-3-hydroxybutyryl.

5. The compound of claim 1, wherein the R² group on the N7 position is acetyl, formyl, or (R)-3-hydroxybutyryl.

6. The compound of claim 1, wherein the linkage between the pseudaminic acid moiety and the linker is a glycosidic linkage.

7. The compound of claim 6, wherein the glycosidic linkage is a or s.

8. The compound of claim 1, wherein the linker is a PEG-based linker with a variable number of (CH₂CH₂O) units, according to formula (II), wherein m is about 1 to about 5:

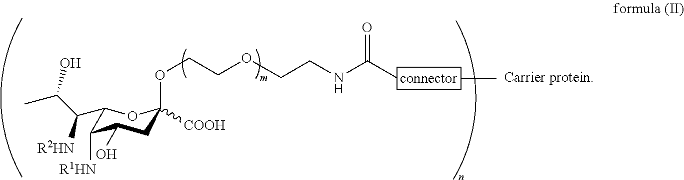

9. The compound of claim 1, wherein the linker is a saturated hydrocarbon chain with variable length of about 2 to about 10, according to formula (III), wherein m is about 0 to about 8:

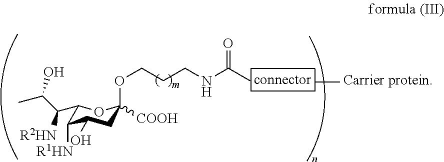

10. The compound of claim 1, wherein the connector is a cyclic lactam structure generated via the ligation between orthophthaldehyde (OPA) moiety and the lysine side chain, according to formula (IV), wherein m is about 0 to about 5:

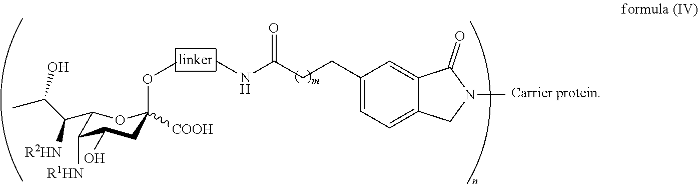

11. The compound of claim 1, wherein the connector is a maleimide thiol adduct generated via Michael addition of thiol to the maleimide modified lysine side chain, according to formula (V), wherein m is about 1 to about 5:

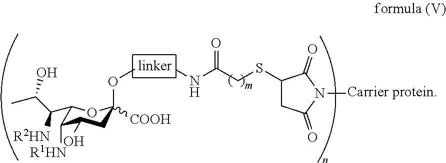

12. The compound of claim 1, wherein the connector is a triazole-based structure generated from the sugar derived alkyne species and azide modified protein side chain, according to formula (VI), wherein m is about 0 to about 5 and p is about 1 to about 5:

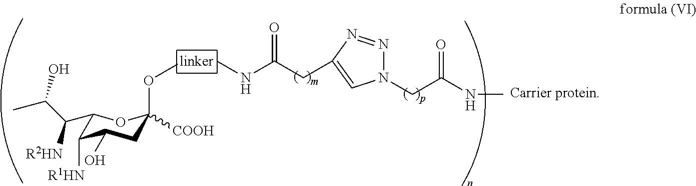

13. The compound of claim 1, wherein the connector is a triazole-based structure generated from the sugar derived azide species and alkyne modified protein side chain, according to formula (VII), wherein m is about 1 to about 5 and p is about 0 to about 5:

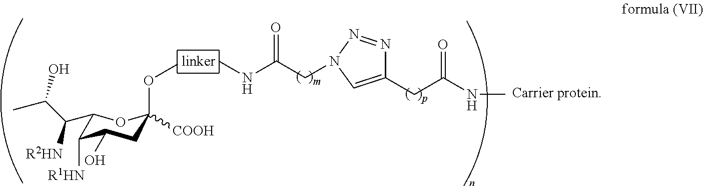

14. The compound of claim 1, wherein the connector is a thiol-alkyne adduct generated via radical addition, according to formula (VIII), wherein m is about 1 to about 5 and p is about 0 to about 5:

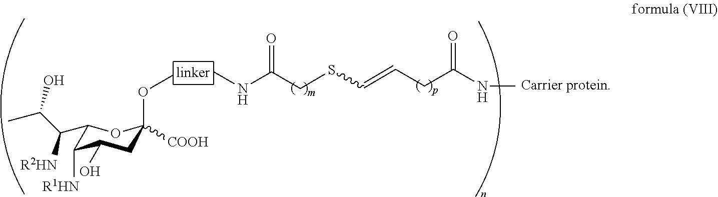

15. The compound of claim 1, wherein the compound is Pse-CRM197 1 (formula (IX)), Pse-CRM197 2 (formula (X)), Pse-CRM197 3 (formula (XI)), or Pse-BSA 17 (formula (XII)):

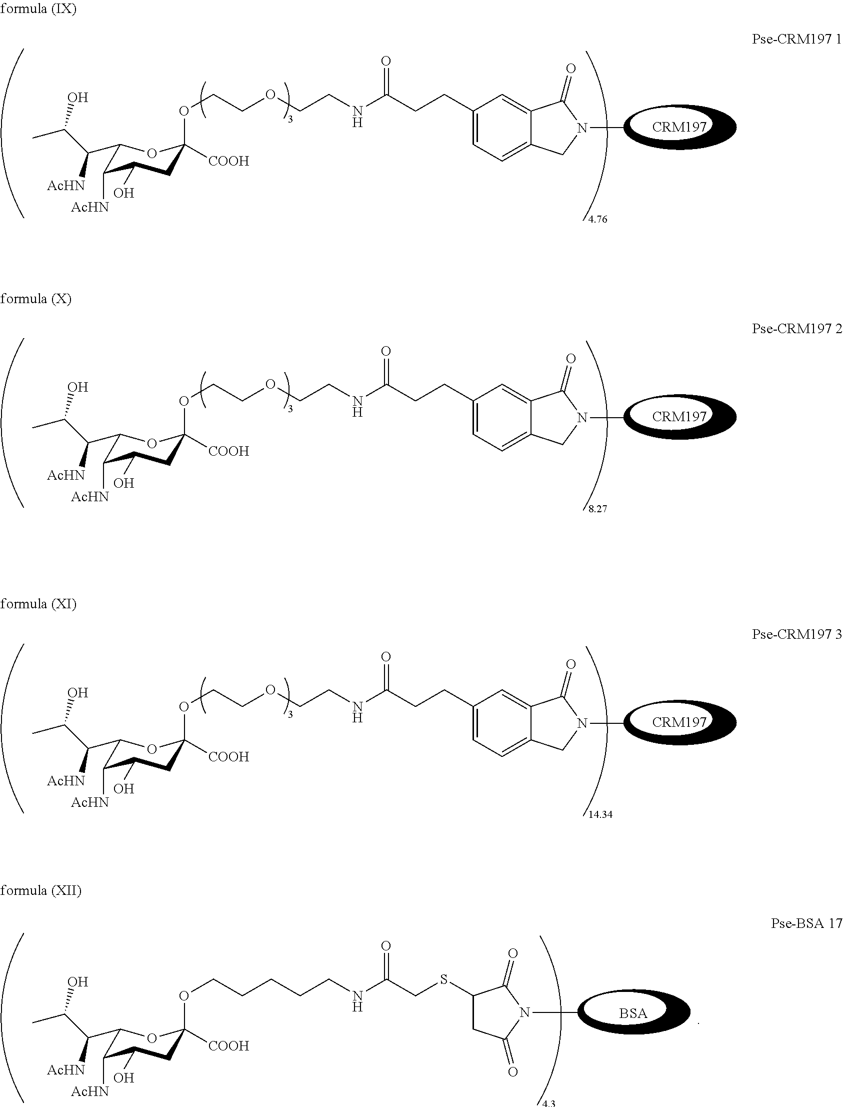

16. A composition comprising the compound of claim 1.

17. The composition of claim 16, further comprising an adjuvant, carrier, excipient, or buffer.

18. A method for raising an immune response in a subject, the method comprising administering an effective amount of the composition of claim 16 to the subject.

19. The method of claim 18, further comprising eliciting the production of IgG1, IgG2b, IgG3, or IgG2c in the subject.

20. A method of inhibiting growth of bacteria in a subject, the method comprising administering an effective amount of the composition of claim 16 to the subject, wherein the bacteria synthesize Pse and bacterial growth is inhibited.

21. The method of claim 20, wherein the bacteria is Acinetobacter spp.

22. The method of claim 21, wherein the Acinetobacter spp. is Acinetobacter baumannii.

23. The method of claim 22, wherein the A. baumannii is A. baumannii strain Ab2.
